(19) United States
(12) Patent Application Publication
(10) Pub. No.: US 2011/0305739 A1
(43) Pub. Date: Dec. 15, 2011

(54) Method for Preparing a Non-Ionic Surfactant Stable Personal Care Dispersion

(76) Inventors: Douglas Allan Royce, Sunman, IN (US); Gregory Thomas Waning, Fairfield, OH (US); Seth Edward Lindberg, West Chester, OH (US); Mark Anthony Brown, Union, KY (US); David Michael Piatt, Orlando, FL (US)

(21) Appl. No.: 13/156,749

(22) Filed: Jun. 9, 2011

Related U.S. Application Data
(60) Provisional application No. 61/352,977, filed on Jun. 9, 2010.

(51) Int. Cl. A61K 8/04; A61K 47/32; A61K 47/34; A61K 9/14; A61P 17/00; A61K 8/81; C11D 17/00; A61Q 5/00; A61Q 19/10; C11D 3/60; A61K 8/84

(52) U.S. Cl. 424/401; 510/137; 510/218; 510/138; 424/49; 510/276; 424/59; 514/18.8; 514/772.3; 514/772.6; 514/772.5; 424/400; 510/158; 510/418; 510/405

(57) ABSTRACT

Disclosed herein are methods for preparing dispersions containing small diameter, liquid crystals of a high charge density cationic polymer and a detersive surfactant. Also disclosed herein are uses of these dispersions to prepare personal care products and liquid cleansing products.

### CROSS REFERENCE TO RELATED APPLICATION

This application claims the benefit of U.S. Provisional Application No. 61/352,977 filed Jun. 9, 2010.

### FIELD OF THE INVENTION

The present invention relates to methods for preparing a dispersion containing small diameter, liquid crystals of a high charge density cationic polymer and a detersive surfactant, and use of these dispersions to prepare personal care products and liquid cleansing products.

### BACKGROUND OF THE INVENTION

Conditioning personal care compositions containing lyotropic liquid crystals are known to reduce the surface energy of hair, thereby increasing its hydrophobicity and restoring its natural, smooth, lubricious feel. Lyotropic liquid crystals are often composed of a detersive surfactant and a high charge density cationic polymer in water. In effect, when a detersive surfactant and high charge density cationic polymer are added together in water, an ionic interaction results between them, which induces areas of liquid crystal formation.

The ability to form liquid crystals composed a high charge density cationic polymer, a detersive surfactant, and water is highly dependent on their relative concentrations. At low concentrations, the cationic polymer and detersive surfactant are randomly dispersed in water without any ordering. At slightly higher concentrations, the detersive surfactant spontaneously assembles into micelles that remain unordered in solution. The cationic polymer can complex with the detersive surfactant to form regions that have higher surfactant concentration complexes within a lower concentration solution. At higher concentration, the assemblies become ordered into different phases of liquid crystals. In the hexagonal phase, the amphiphiles form long cylinders that are arranged into a hexagonal lattice. In the lamellar phase, the amphiphiles arrange themselves into extended sheets that are separated by thin layers of water.

The size of the liquid crystals that form in the conditioning personal care compositions is also important. Liquid crystals with small diameters (e.g., less than about 100 μm, and in one embodiment less than about 20 μm) are useful. Liquid crystals with larger diameters (e.g., greater than about 100 μm) will contain macroscopic particles that affect the deposition profile, consumer perception, experience, and aesthetic qualities of the final personal care product. The size of the liquid crystals depends on the nature of the charge-charge interaction that occurs when the high charge density cationic polymer is introduced to the detersive surfactant. A rapid charge-charge interaction between the high charge density cationic polymer and detersive surfactant results in liquid crystals with undesirable, large diameters, while a slow charge-charge interaction results in liquid crystals with desirable, small diameters. The rate of charge-charge interaction between the cationic polymer and detersive surfactant is determined by a delicate interplay between the order, rate, and energy of addition of the cationic polymer and detersive surfactant. Because so many parameters influence the rate charge-charge interaction, controlling it has traditionally proved especially difficult.

Thus, every time a new conditioning personal care product is formulated, the parameters controlling liquid crystal formation must be reoptimized, resulting in significant time and cost disadvantages.

### SUMMARY OF THE INVENTION

Disclosed herein are methods for forming a dispersion containing liquid crystals of a high charge density cationic polymer and a detersive surfactant at ambient temperature (about 20° C. to about 40° C.). In this method, a synthetic, cationic polymer that has a cationic charge density of at least about 2 molar equivalents per gram (meq/g), in one embodiment at least about 5 meq/g, for example, about 7 meq/g, is added to a neutralized, preserved, detersive surfactant at a rate sufficient to provide an instantaneous weight ratio of cationic polymer to forming dispersion of about 1:10 to about 1:100, in one embodiment from about 1:12.5 to about 1:50, in another embodiment from about 1:15 to about 1:30. Further, during addition of the cationic polymer the forming dispersion has an energy of about 10 Joules per kilogram (J/kg) to about 10,000 J/kg, in one embodiment from about 100 J/kg to about 7,500 J/kg, in another embodiment from about 500 J/kg to about 5000 J/kg. The resulting dispersion can have a final cationic polymer concentration of about 1 wt. % to about 10 wt. %, in one embodiment from about 3 wt. % to about 6 wt. %, for example, about 4 wt. % of the cationic polymer, based on the total weight of the dispersion. Optionally, the resulting dispersion is stabilized from settling and creaming through the addition of a suspending agent. The liquid crystals that form in the dispersion have an average diameter of less than about 100 μm, in one embodiment from about 1 μm to about 20 μm.

Another aspect of the invention is a method for making a personal care product. In this method, the dispersion containing liquid crystals of a high charge density cationic polymer and a detersive surfactant are added to a personal care composition that contains a component selected from the group consisting of conditioning agents, natural cationic deposition polymers, synthetic cationic deposition polymers, anti-dandruff agents, gel networks (e.g., fatty alcohol/surfactant networks), particles, suspending agents, paraffinic hydrocarbons, propellants, viscosity modifiers, dyes, non-volatile solvents, water soluble diluents, water insoluble diluents, pearlescent aids, foam boosters, surfactants, pediculocides, pH adjusting agents, perfumes, preservatives, chelants, proteins, skin active agents, sunscreens, UV absorbers, vitamins, and mixtures thereof.

Another aspect of the invention is a method for making a liquid cleansing product. In this method, the dispersion containing liquid crystals of a high charge density cationic polymer and a detersive surfactant are added to a liquid cleansing composition that contains a component selected from the group consisting of diamines, organic solvents, polycarboxylate polymers, magnesium ions, hydrotropes, polymer suds stabilizers, carboxylic acids, detersive enzymes, optical brighteners, dye transfer inhibition agents, suds suppressors, detersive soil release polymers, fabric care benefit agents, stabilizers, ancillary detersive surfactants, detersive builders, perfumes, coloring agents, enzymes, bleaches, mal-odor control agents, antimicrobials, anti-static agents, fabric softening agents, grease cleaning polymers, and mixtures thereof.

Additional features of the invention may become apparent to those skilled in the art from a review of the following detailed description, taken in conjunction with the drawings, examples and the appended claims.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 depicts an image of a personal care product, taken using Nomarski differential interference contrast (DIC) microscopy, to which the dispersion of the invention has been incorporated. The larger particles (e.g., about 2 μm to about 10 μm in diameter) are liquid crystals of the cationic polymer, diallyldimethyl ammonium chloride (DADMAC, Polyquaternium-6) and a surfactant, and the smaller, feather-like particles (e.g., about 2 μm in length) are ethylene glycol distearate (EGDS) crystals.

FIG. 2 represents a process for production of a dispersion containing liquid crystals of a high charge density cationic polymer and detersive surfactant using a high energy mill.

FIG. 3 represents a process for production of a dispersion containing liquid crystals of a high charge density cationic polymer and detersive surfactant using a high energy mill, further using a static mixer to blend in stabilizer.

FIG. 4 represents a process for production of a dispersion containing liquid crystals of a high charge density cationic polymer and detersive surfactant using a Liquid Whistle device.

FIG. 5 represents a process for production of a dispersion containing liquid crystals of a high charge density cationic polymer and detersive surfactant using a Liquid Whistle device, further using a static mixer to blend in stabilizer.

### DETAILED DESCRIPTION OF THE INVENTION

A method has been found for the preparation of a dispersion containing liquid crystals of a detersive surfactant and a high charge density cationic polymer in water. The liquid crystals have average diameters of less than about 100 μm, and in one embodiment from about 1 μm to about 20 μm. The liquid crystal dispersion of the invention is advantageous because it is phase stable (i.e., the detersive surfactant and/or the cationic polymer do not crystallize out of solution at about 20° C. to about 40° C.), has a high active concentration of cationic polymer and detersive surfactant, can be preserved, can be prepared at ambient temperature, and is inexpensive. Further, the dispersion of the invention is easily dispersible in personal care compositions using a large variety of process conditions (e.g., batch process, continuous process etc).

The term “liquid crystal”, as used herein, means a material having phases that are ordered and/or crystalline in only one or two of their three possible orthogonal directions and are disordered (random and/or liquid-like) in the other dimensions.

The term “lyotropic”, as used herein, means that the ordering effects of a material are induced by changing both its concentration and temperature.

The term “polymer”, as used herein, shall include materials whether made by polymerization of one type of monomer or made by two (i.e., copolymers) or more types of monomers.

The term “water soluble”, as used herein, means that the polymer is soluble in water in the present composition. In general, the polymer should be soluble at 25° C. at a concentration of 0.1 wt. %, in one embodiment at 1 wt. %, in another embodiment at 5 wt. %, and in yet another embodiment at 15 wt. %, based on the weight of the water solvent.

The term “charge density” as used herein, means the ratio of the number of positive charges on a monomeric unit of which a polymer is comprised to the molecular weight of said monomeric unit. The charge density multiplied by the polymer molecular weight determines the number of positively charged sites on a given polymer chain.

The term “alkyl” refers to a saturated or unsaturated straight or branched chain hydrocarbon group of carbon atoms, including, but not limited to, methyl, ethyl, n-propyl, isopropyl, n-butyl, isobutyl, tert-butyl, n-hexyl, and the like. C₁₋₈alkyl refers to substituted or unsubstituted alkyl groups that can have, for example from 1 to 8 carbon atoms. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halogen, aryl, heteroaryl, cycloalkyl, heterocycloalkyl, and amino. “Heteroalkyl” is defined similarly as alkyl, except the heteroalkyl contains at least one heteroatom independently selected from the group consisting of oxygen, nitrogen, and sulfur.

The term “alkylene” refers to a straight or branched alkyl group chain having two points of attachment to the rest of the molecule.

The term “alkenyl” refers to a straight or branched chain hydrocarbon group of at least two carbon atoms containing at least one carbon double bond including, but not limited to, 1-propenyl, 2-propenyl, 2-methyl-1-propenyl, 1-butenyl, 2-butenyl, and the like.

The term “alkylidene” refers to a straight or branched alkene group having two points of attachment to the rest of the molecule.

The term “alkoxy” refers to a straight or branched chain alkyl group covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “oxyalkylene” refers to an alkoxy group having two points of attachment to the rest of the molecule, one of the points being through the oxygen atom.

The term “alkoxyalkyl” refers to one or more alkoxy groups appended to an alkyl group.

The term “aryl” refers to a monocyclic or polycyclic aromatic group, in one embodiment a monocyclic or bicyclic aromatic group, e.g., phenyl or naphthyl. Unless otherwise indicated, an aryl group can be unsubstituted or substituted with one or more, and in particular one to five groups independently selected from, for example, halogen, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

The term “heteroaryl” refers to a monocyclic or polycyclic aromatic group, in one embodiment a monocyclic or bicyclic aromatic group, containing at least one nitrogen, oxygen, or sulfur atom in an aromatic ring. Unless otherwise indicated, a heteroaryl group can be unsubstituted or substituted with 1 to 5 groups. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

The term “alkylaryl” refers to one or more alkyl groups appended to an aryl group.

The term “alkoxyaryl” refers to one or more alkoxy groups appended to an aryl group.

The term “arylalkyl” refers to one or more aryl groups appended to an alkyl group.

The term “aryloxy” refers to an aromatic group covalently bonded to the parent molecule through an —O— linkage.

The term “alkylaryloxy” refers to an alkylaryl group covalently bonded to the parent molecule through an —O— linkage.

The term “alkanol” refers to a straight or branched chain alkyl group covalently bonded to OH.

The term “alkanolamine” refers to straight or branched chain alkyl groups covalently bonded to a hydroxy moiety and to a amino moiety. Examples of alkanolamine include propanolamine, ethanolamine, dimethylethanolamine, and the like.

The term “amido” refers to a group having a NH₂ radical that is bonded to a C═O radical.

The term “alkanolamide” refers to a straight or branched chain alkyl group covalently bonded to a hydroxy moiety and to an amide moiety.

The term “alkylsulfate” refers to a straight or branched chain alkyl group covalently to SO₃⁻.

The term “benzyl” refers to a benzene radical that can be unsubstituted or substituted with one or more, and in particular one to five groups independently selected from, for example, halogen, alkyl, alkenyl, OCF₃, NO₂, CN, NC, OH, alkoxy, amino, CO₂H, CO₂alkyl, aryl, and heteroaryl.

The term “halogen” or “halo” refers to fluoro, chloro, bromo, or iodo.

All percentages, parts and ratios are based upon the total weight of the compositions of the present invention, unless otherwise specified. All such weights as they pertain to listed ingredients are based on the active level and therefore do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

In one aspect, the invention relates to a method for forming a dispersion containing liquid crystals in water. In this method, a synthetic, cationic polymer with a cationic charge density of at least about 2 molar equivalents per gram (meq/g), in one embodiment at least about 5 meq/g, for example, about 7 meq/g is added to a neutralized, preserved, detersive surfactant, to form dispersion comprising liquid crystals. The resulting dispersion can have a final cationic polymer concentration of about 1 wt. % to about 10 wt. %, in one embodiment about 3 wt. % to about 6 wt. %, in another embodiment about 4 wt. % of the cationic polymer, based on the total weight of the dispersion. Optionally, the dispersion is then stabilized to prevent it from separating into multiple layers. The cationic polymer is added to the detersive surfactant at a rate sufficient to provide an instantaneous weight ratio of cationic polymer to forming dispersion of about 1:10 to about 1:100, in one embodiment from about 1:12.5 to about 1:50, in another embodiment from about 1:15 to about 1:30. Further, during addition of the cationic polymer the forming dispersion has an energy of about 10 Joules per kilogram (J/kg) to about 10,000 J/kg, in one embodiment from about 100 J/kg to about 7,500 J/kg, in another embodiment about 500 J/kg to about 5,000 J/kg. The resulting liquid crystals have average diameters of less than about 100 μm, in one embodiment from about 1 μm to about 20 μm. The method of the invention can occur at a temperature of about 20° C. to about 99° C., in one embodiment from about 20° C. to about 75° C., in another embodiment from about 20° C. to about 40° C.

The detersive surfactant provides cleaning performance to the dispersion and aids in the formation of the liquid crystal phase. The detersive surfactant contains at least one anionic surfactant, which has an ethyoxylate level of about 0 to about 10 and an anion level of about 1 to about 10, and optionally an amphoteric surfactant, a zwitterionic surfactant, a cationic surfactant, a nonionic surfactant, or a combination thereof. Such surfactants should be physically and chemically compatible with the essential components described herein, or should not otherwise unduly impair product stability, aesthetics, or performance.

An optimal ethoxylate level is calculated based on the stoichiometry of the surfactant structure, which in turn is based on a particular molecular weight of the surfactant where the number of moles of ethoxylation is known. Likewise, given a specific molecular weight of a surfactant and an anionization reaction completion measurement, the anion level can be calculated. Analytical techniques have been developed to measure ethoxylation or anionization within surfactant systems.

The Level of Ethoxylate and the Level of Anion representative of a particular surfactant system are calculated from the percent ethoxylation and percent anion of individual surfactants in the following manner. The Level of Ethoxylate is equal to the percent ethoxylation multiplied by percent active ethoxylated surfactant (based on the total weight of the dispersion). The Level of Anion is equal to the percent anion in ethyoxylated surfactant multiplied by percent active ethoxylated surfactant (based on the total weight of the dispersion) plus percent anion in non-ethoxylated surfactant (based on the total weight of the dispersion). If a dispersion comprises two or more surfactants having different respective anions (e.g., surfactant A has a sulfate group and surfactant B has a sulfonate group), the Level of Anion in the dispersion is the sum of the molar levels of each respective anion as calculated above.

For example, a detersive surfactant contains 48.27 wt. % 3-ethoxylated sodium laureth sulfate (SLE3S) and 6.97 wt. % sodium lauryl sulfate (SLS), based on the total weight of the dispersion. The ethoxylated surfactant (SLE3S) contains 0.294321% ethoxylate and 0.188307% sulfate as the anion and the non-ethoxylated surfactant (SLS) contains 0.266845% sulfate as an anion. Because both of the SLE and SLS are about 29% active, the detersive surfactant contains about 14 wt. % of active SLE3S and about 2 wt. % of active SLS, based on the total weight of the dispersion. The Level of Ethoxylate is 0.294321 multiplied by 14 (% active ethoxylated surfactant). Thus, the Level of Ethoxylate in this example detersive surfactant is 4.12. The Level of Anion is 0.188307 multiplied by 14 (% active ethoxylated surfactant) plus 0.266845 multiplied by 2 (% active non-ethoxylated surfactant). Thus the Level of Anion in this example detersive surfactant is 3.17.

In one embodiment, the detersive surfactant includes at least one anionic surfactant that contains an anion selected from the group consisting of sulfates, sulfonates, sulfosuccinates, isethionates, carboxylates, phosphates, and phosphonates. In one embodiment the anion is a sulfate. Other potential anions for the anionic surfactant include phosphonates, phosphates, and carboxylates.

Anionic surfactants suitable for use in the dispersions are alkyl sulfates and alkyl ether sulfates. These materials have the respective formulae ROSO₃M and RO(C₂H₄O)ₓSO₃M, wherein R is alkyl or alkenyl of about 8 to about 18 carbon atoms, x is an integer having a value of from about 1 to about 10, and M is a cation such as ammonium, an alkanolamine such as triethanolamine, a monovalent metal cation such as sodium and potassium, or a polyvalent metal cation such as magnesium and calcium. Solubility of the surfactant will depend upon the particular anionic surfactants and cations chosen.

In one embodiment, R has about 8 to about 18 carbon atoms, in another embodiment from about 10 to about 16 carbon atoms, in yet another embodiment from about 12 to about 14 carbon atoms, in both the alkyl sulfates and alkyl ether sulfates. The alkyl ether sulfates are typically made as condensation products of ethylene oxide and monohydric alcohols having about 8 to about 24 carbon atoms. The alcohols can be synthetic or they can be derived from fats, e.g., coconut oil, palm kernel oil, tallow. In one embodiment the lauryl alcohol and straight chain alcohols are derived from coconut oil or palm kernel oil. Such alcohols are reacted with about 0 to about 10, in one embodiment from about 0 to about 5, in another embodiment from about 0, 1 or 3, molar proportions of ethylene oxide, and the resulting mixture of molecular species having, for example, an average of 0, 1, or 3 moles of ethylene oxide per mole of alcohol is sulfated and neutralized.

Specific non-limiting examples of alkyl ether sulfates which may be used in the dispersion of the invention include sodium and ammonium salts of coconut alkyl triethylene glycol ether sulfate, tallow alkyl triethylene glycol ether sulfate, and tallow alkyl hexa-oxyethylene sulfate. In one embodiment the alkyl ether sulfates are those comprising a mixture of individual compounds, wherein the compounds in the mixture have an average alkyl chain length of about 10 to about 16 carbon atoms and an average degree of ethoxylation of about 1 to about 4 moles of ethylene oxide. Such a mixture also comprises about 0 wt. % to about 20 wt. % of C₁₂₋₁₃ compounds; about 60 wt. % to about 100 wt. % of C₁₄₋₁₆ compounds; about 0 wt. % to about 20 wt. % of C₁₇₋₁₉ compounds; about 3 wt. % to about 30 wt. % of compounds having a degree of ethoxylation of 0; about 45 wt. % to about 90 wt. % of compounds having a degree of ethoxylation about 1 to about 4; about 10 wt. % to about 25 wt. % of compounds having a degree of ethoxylation of about 4 to about 8; and about 0.1 wt. % to about 15 wt. % by weight of compounds having a degree of ethoxylation greater than about 8, based on the total weight of the alkyl ether sulfate.

Suitable anionic detersive surfactant components include those which are known for use in hair care or other personal care cleansing compositions. In one embodiment the anionic detersive surfactants components for use in the dispersion of the invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

In some embodiments, the detersive surfactant further includes one or more additional surfactants selected from the group consisting of amphoteric surfactants, zwitterionic surfactants, cationic surfactants, nonionic surfactants, and mixtures thereof. These surfactants are known for use in hair care or other personal care cleansing compositions and contain a group that is anionic at the pH of the dispersion. The concentration of such amphoteric detersive surfactants in these personal cleansing compositions ranges from about 0.5 wt. % to about 20 wt. %, in one embodiment from about 1 wt. % to about 10 wt. %, based on the total weight of the cleansing composition. Non-limiting examples of suitable zwitterionic or amphoteric surfactants are described in U.S. Pat. Nos. 5,104,646 and 5,106,609.

Amphoteric surfactants suitable for use in the dispersion of the invention include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. In one embodiment the amphoteric surfactants for use in the dispersions comprise cocoamphoacetate, cocoamphodiacetate, lauroamphoacetate, lauroamphodiacetate, lauramine oxide, and mixtures thereof.

Zwitterionic surfactants suitable for use include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines (i.e., cocoamidopropyl betaine, cocobetaine).

The dispersion of the invention may further comprise additional surfactants for use in combination with the detersive surfactant component described herein. Other suitable anionic surfactants are the water-soluble salts of organic, sulfuric acid reaction products conforming to the formula [R¹—SO₃-M] where R′ is a straight or branched chain, saturated, aliphatic hydrocarbon radical having from about 8 to about 24, in one embodiment from about 10 to about 18 carbon atoms; and M is a cation, as previously described herein. Nonlimiting examples of such surfactants are the salts of an organic sulfuric acid reaction product of a hydrocarbon of the methane series, including iso-, neo-, and n-paraffins, having from about 8 to about 24 carbon atoms, in one embodiment from about 12 to about 18 carbon atoms and a sulfonating agent, e.g., SO₃, H₂SO₄, obtained according to known sulfonation methods, including bleaching and hydrolysis. In another embodiment alkali metal and ammonium sulfonated C₁₀₋₁₈ n-paraffins.

Other suitable anionic surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil.

Other anionic surfactants suitable for use in the dispersion of the invention are the succinates, examples of which include disodium N-octadecylsulfosuccinate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 10 to about 24 carbon atoms. In this context, the term “olefin sulfonates” refers to compounds which can be produced by the sulfonation of alpha-olefins by means of uncomplexed sulfur trioxide, followed by neutralization of the acid reaction mixture in conditions such that any sulfonates which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form. The alpha-olefins from which the olefin sulfonates are derived are mono-olefins having about 10 to about 24 carbon atoms, in one embodiment about 12 to about 16 carbon atoms. In another embodiment, they are straight chain olefins. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A nonlimiting example of such an alpha-olefin sulfonate mixture is described in U.S. Pat. No. 3,332,880.

Another class of anionic surfactants suitable for use in the dispersions are the beta-alkyloxy alkane sulfonates. These surfactants conform to the below formula:

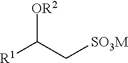

where R¹ is a straight chain alkyl group having about 6 to about 20 carbon atoms, R² is a lower alkyl group having about 1 to about 3 carbon atoms, in one embodiment 1 carbon atom, and M is a water-soluble cation, as previously described herein. Suitable anionic surfactants for use in the dispersion of the invention include sodium tridecyl benzene sulfonate, sodium dodecyl benzene sulfonate, and mixtures thereof.

Amides, including alkanolamides, are the condensation products of fatty acids with primary and secondary amines or alkanolamines to yield products of the general below formula:

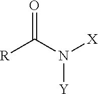

wherein RCO is a fatty acid radical and R is C₈₋₂₀; X is an alkyl, aromatic or alkanol (CHR′CH₂OH wherein R′ is H or C₁₋₆ alkyl); Y is H, alkyl, alkanol or aromatic. Suitable amides include, but are not limited to cocamide, lauramide, oleamide and stearamide. Suitable alkanolamides include, but are not limited to, cocamide DEA, cocamide MEA, cocamide MIPA, isostearamide DEA, isostearamide MEA, isostearamide MIPA, lanolinamide DEA, lauramide DEA, lauramide MEA, lauramide MIPA, linoleamide DEA, linoleamide MEA, linoleamide MIPA, myristamide DEA, myristamide MEA, myristamide MIPA, Oleamide DEA, Oleamide MEA, Oleamide MIPA, palmamide DEA, palmamide MEA, palmamide MIPA, palmitamide DEA, palmitamide MEA, palm kernelamide DEA, palm kernelamide MEA, palm kernelamide MIPA, peanutamide MEA, peanutamide MIPA, soyamide DEA, stearamide DEA, stearamide MEA, stearamide MIPA, tallamide DEA, tallowamide DEA, tallowamide MEA, undecylenamide DEA, undecylenamide MEA, PPG-2 Hydroxyethyl cocoamide, and PPG-2-Hydroxyethyl Coco/Isostearamide. The condensation reaction may be carried out with free fatty acids or with all types of esters of the fatty acids, such as fats and oils, and particularly methyl esters. The reaction conditions and the raw material sources determine the blend of materials in the end product and the nature of any impurities.

Suitable optional surfactants include nonionic surfactants. Any such surfactant known in the art for use in hair or personal care products may be used, provided that the optional additional surfactant is also chemically and physically compatible with the essential components of the dispersion of the invention, or does not otherwise unduly impair product performance, aesthetics or stability. The concentration of the optional additional surfactants in the dispersion may vary with the cleansing or lather performance desired, the optional surfactant selected, the desired product concentration, the presence of other components in the dispersion, and other factors well known in the art.

Nonlimiting examples of other surfactants suitable for use in the dispersions are described in McCutcheon's, Emulsifiers and Detergents, 1989 Annual, published by M.C. Publishing Co., and U.S. Pat. Nos. 3,929,678; 2,658,072; 2,438,091; 2,528,378.

The cationic polymer described herein aids in conditioning hair. For example, the cationic polymer can provide damaged hair or particularly chemically treated hair, with a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when liquid crystals are applied to chemically treated hair, the hair becomes more hydrophobic and more virgin-like, in both look and feel. Without being limited to any theory, it is believed that the liquid crystal complex creates a hydrophobic layer or film, which coats the hair fibers and protects the hair, much like the natural F-layer protects the hair. The hydrophobic layer returns the hair to a generally virgin-like, healthier state.

The liquid crystals of the dispersion of the invention are formed by combining the synthetic cationic polymers described herein with the aforementioned anionic detersive surfactant component of the dispersion. The synthetic cationic polymer has a relatively high charge density. It should be noted that some synthetic polymers having a relatively high cationic charge density do not form liquid crystals, primarily due to their abnormal linear charge densities. Such synthetic cationic polymers are described in WO 94/06403 to Reich et al. The synthetic polymers described herein can be formulated in a stable dispersion that provides improved conditioning performance. In some embodiments, the synthetic cationic polymer may be formed from:

i) one or more cationic monomer units, and optionally

ii) one or more monomer units bearing a negative charge, and/or

iii) a nonionic monomer,

wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers.

The cationic polymers have a cationic charge density of about 2 molar equivalents per gram (meq/g) to about 7 meq/g, in one embodiment from about 3 meq/g to about 7 meq/g, in another embodiment from about 4 meq/g to about 7 meq/g. In some embodiments, the cationic charge density is about 6.2 meq/gm. The cationic polymers also have a molecular weight of about 1,000 to about 5,000,000, in one embodiment from about 10,000 to about 2,000,000, in another embodiment from about 100,000 to about 2,000,000.

In one embodiment, the cationic polymers are water soluble or dispersible, non-crosslinked, synthetic cationic polymers having the following structure:

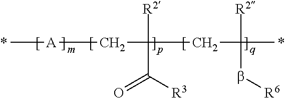

m>1

p=0 or >1

q=0 or >1

m>p

wherein A may be one or more of the following cationic moieties:

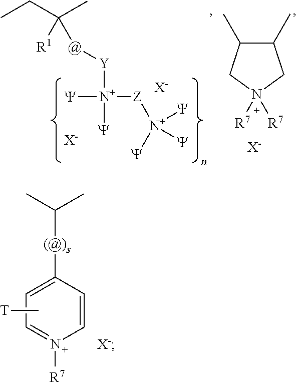

@ is amido, alkylamido, ester, ether, alkyl or alkylaryl;

Y is C₁₋₂₂ alkyl, alkoxy, alkylidene, alkyl or aryloxy;

Ψ is C₁₋₂₂ alkyl, alkyloxy, alkyl aryl or alkyl aryloxy;

Z is C₁₋₂₂ alkyl, alkyloxy, aryl or aryloxy;

R¹ is H, C₁₋₄ linear or branched alkyl;

s is 0 or 1;

n is 0 or ≧1;

T is C₁₋₂₂ alkyl;

R⁷ is C₁₋₂₂ alkyl; and,

X is halogen, hydroxide, alkoxide, sulfate or alkylsulfate.

The monomer bearing a negative charge is defined by R^{2′} is H, C₁₋₄ linear or branched alkyl and R³ is:

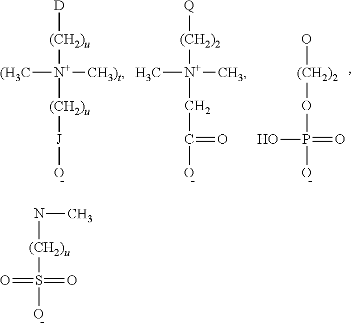

wherein D is O, N, or S;

Q is NH₂ or O;

u is 1-6;

t is 0-1; and,

J is an oxygenated functional group containing the following elements P, S, C.

The nonionic monomer is defined by R^{2″} is H, C₁₋₄ linear or branched alkyl, R⁶ is linear or branched alkyl, alkyl aryl, aryloxy, alkyloxy, alkylaryloxy and β is defined as

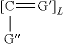

wherein G′ and G″ are, independently of one another, O, S or N—H and L is 0 or 1.

Examples of cationic monomers include aminoalkyl(meth)acrylates, (meth)aminoalkyl(meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl(meth)acrylate, dimethylaminopropyl(meth)acrylate, ditertiobutylaminoethyl(meth)acrylate, dimethylaminomethyl(meth)acrylamide, dimethylaminopropyl(meth)acrylamide, ethylenimine, vinylamine, 2-vinylpyridine, 4-vinylpyridine, trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride, diallyldimethyl ammonium chloride.

Cationic monomers comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

In one embodiment cationic monomers include trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride.

In yet another embodiment the cationic monomers include trimethyl ammonium propyl(meth)acrylamido chloride (MAPTAC), diallyldimethyl ammonium chloride (DADMAC, Polyquaternium-6), and mixtures thereof.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

In one embodiment monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide(meth)acrylate (i.e. polyethoxylated(meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrrolidone, and vinyl aromatic compounds.

In yet another embodiment the nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X⁻) in association with the synthetic cationic polymers may be any known counterion so long as the polymers remain soluble or dispersible in water, in the dispersion, and so long as the counterions are physically and chemically compatible with the essential components of the dispersion or do not otherwise unduly impair product performance, stability or aesthetics. Nonlimiting examples of such counterions include halides (e.g., chlorine, fluorine, bromine, iodine), sulfate and methylsulfate.

The amount of synthetic cationic polymer used is dependent on the molar ratio of cationic polymer to detersive surfactant and on the number of charges on each monomer unit. In general, the detersive surfactant should have a molar ratio to the cationic polymer that is at least 1 mole of detersive surfactant times the number of charges on the cationic monomer unit, to 1 mole of cationic polymer, as shown below:

(>1 mole of detersive surfactant) (# of charges on the cationic monomer unit of the cationic polymer): (1 mole of the cationic polymer).

For example, if each cationic monomer unit of the cationic polymer has one charge, then the ratio of detersive surfactant to cationic polymer is at least 1:1. If each cationic monomer unit of the cationic polymer has two charges, then the ratio of detersive surfactant to cationic polymer is at least 2:1. If each cationic monomer unit of the cationic polymer has three charges, then the ratio of detersive surfactant to cationic polymer is at least 3:1.

In the below embodiments, the # of charges on the cationic monomer unit of the cationic polymer is represented by ‘C.’

In some embodiments, the molar ratio of detersive surfactant to cationic polymer is about 1C:1 to about 20C:1, in another embodiment from about 1C:1 to about 5C:1, in another embodiment from about 2C:1

In some embodiments the dispersion can include about 1 wt. % to about 10.0 wt. %, in one embodiment from about 1.5 wt. % to about 9.0 wt. %, in another embodiment about 3 wt. % to about 6 wt. %, in yet another embodiment from about 4 wt. % of cationic polymer, and in yet another embodiment from about 5 wt. % to about 30 wt. % of detersive surfactant, based on the total weight of the dispersion.

As previously described, the detersive surfactant, synthetic cationic polymer, and water are at concentrations that allow the dispersion to exist as liquid crystals. In some embodiments, the molar ratio of cationic polymer to water is from about 1:50 to about 1:1000, in one embodiment from about 1:50 to about 1:250, in yet another embodiment from about 1:150. Thus, in some embodiments, the molar ratio of detersive surfactant to cationic polymer to water is about 1C:1:50 to about 20C:1:50, or about 1C:1:1000 to about 20C:1:1000. In one embodiment the molar ratio of detersive surfactant to cationic polymer to water is about 1C:1:50 to about 5C:1:50, or about 1C:1:250 to about 5C:1:250. For example, the molar ratio of detersive surfactant to cationic polymer to water can be about 2C:1:150.

In some embodiments of the invention, the detersive surfactant includes sodium lauryl sulfate (SLS). In these embodiments, the active weight ratio of SLS to cationic polymer is about 2.5:1.

In some embodiments of the invention, the detersive surfactant includes sodium laureth sulfate (e.g., sodium laureth sulfate 1-ethoxylate (SLE1S) or sodium laureth sulfate 3-ethoxylate (SLE3S)). In these embodiments, the active weight ratio of detersive surfactant to cationic polymer is about 3.5:1.

In some embodiments of this aspect of the invention, the neutralized, preserved, detersive surfactant can be prepared by: (i) adding a detersive surfactant to water; (ii) adding a preservative to the detersive surfactant to form a preserved, detersive surfactant; and (iii) adding acid to the preserved, detersive surfactant to form a neutralized, preserved, detersive surfactant. The preservative and acid can be added to the detersive surfactant at any rate and at a temperature of about 20° C. to about 99° C., in one embodiment about 20° C. to about 75° C., in yet another embodiment from about 20° C. to about 40° C.

The preservative can be any preservative that is compatible with the components in the dispersion. Nonlimiting examples of preservatives include sodium benzoate, benzyl alcohol, potassium sorbate, disodium ethylene tetraacetate (Na₂EDTA), tetrasodium ethylenediamine tetraacetate (Na₄EDTA), and mixtures thereof. The preservative can be present in any amount that is effective for killing or inhibiting the growth of microorganisms. The amount of preservative is dependent on the specific preservative used. For example, the preservative can include at least about 0.25 wt. % sodium benzoate, about 5 ppm to about 15 ppm of methylchloroisothiazolinone (KATHON®) or mixtures thereof, based on the total weight of the dispersion. Additional examples of preservatives useful for the dispersion of the invention and amounts (based on the total weight of the dispersion) are listed in Table 1.

TABLE 1
Preservatives

| | | Amount (based on the total |
|---|---|---|
| Class | Name | weight of a dispersion) |
| Formaldehyde | Formaldehyde | 0.05 wt. % to 0.2 wt. % |
| and/or | Germall 115 imidazolidinyl urea | 0.05 wt. to 0.5 wt. % |
| Formaldehyde | [N-methylenebis-(N-1-(hydroxymethyl)-2,5- | |
| Donors | dioxo-4-imidazolidinyl) urea] | |
| | Germall II diazolidinyl urea | 0.03 wt. % to 0.3 wt. % |
| | [N-(hydroxymethyl)-N-(1,3- | |
| | dihydroxymethyl-2,5-dioxo-4- | |
| | imidazolidinyl-n(hydroxymethyl) urea] | |
| | DOWICIL ® 200 (Quaterium 15) | 0.02 wt. % to 0.3 wt. % |
| | [cis-isomer of 1-(3-chloroallyl)-3,5,7-triaza- | |
| | 1-azoniaadamantane chloride] | |
| | GLYDANT ® (1,3-Bis(hydroxymethyl)-5,5- | 0.15 wt. % to 0.4 wt. % |
| | dimethylhydantoin) or other dimethylol | |
| | dimethyl hydantoin (DMDMH) actives | |
| | Glutaraldehyde | 0.01 wt. % to 0.1 wt. % (active) |
| | Bronopol 2-bromo-2-nitropropane-1,3 diol | 0.01 wt. % to 0.1 wt. % |
| Parabens | Hydroxybenzoates (methyl, ethyl, propyl, | less than or equal to 0.3 wt. % |
| | butyl, isobutyl) | |
| Isothiazolinones | 2-Methyl-4-isothiazoline-3-one | 50 ppm to 100 ppm (active) |
| | 5-Chloro-2-methyl-4-isothiazoline-3-one | typically used in combination |
| | | with 2-methyl-4-isothiazoline- |
| | | 3-one |
| | Isothiazolinone | 5 ppm to 15 ppm (active), |
| | | 0.033 wt. % to 0.1 wt. % (as |
| | | added) |
| Organic acids | Dehydroacetic acid (3-acetyl-6-methyl-2H- | 0.02 wt. % to 0.2 wt. % |
| | pyran-2,4-(3H)-dione) and its salts | |
| | Sorbic acid (2,4-hexadienoic acid) and its | 0.025 wt. % to 0.2 wt. % |
| | salts | |
| | Benzoic acid and its salts | 0.1 wt. % to 0.2 wt. % |
| | Citric acid and salts | amount necessary to reach pH |
| | Propionic acid and salts | up to 1 wt. % |
| | Boric acid | 0.01 wt. % to 1 wt. % |
| | Salicylic acid (ortho-hydroxybenzoic acid) | 0.1 wt. % to 0.5 wt. % |
| Alcohols | Benzyl alcohol | 0.25 wt. % 3 wt. % |
| | Chlorobutanol | up to 0.5 wt. %% |
| | Dichlorobenzyl alcohol | 0.05 wt. % to 0.5 wt. % |
| | Phenethyl alcohol (2-phenylethanol) | 1 wt. % to 2 wt. % |
| | 2-Phenoxyethanol | 0.5 wt. % 1 wt. % |
| | Ethanol | 10 wt. % or more |
| Phenolics | Chloroxylenol (para-chloro-2,5- | 0.2 wt. % to 0.8 wt. % |
| | dimethylphenol) | |
| | ortho-Phenylphenol | 0.05 wt. % to 0.5 wt. % |
| Pyrithiones | Zinc pyrithione (zinc-bis-(2-pyridinethiol-2- | 250 ppm to 1000 ppm (active) |
| | oxide) | |
| | Sodium pyrithione (sodium 2-pyridinethiol- | 250 ppm to 1000 ppm (active) |
| | 1-oxide) | |

The acid functions to neutralize the dispersion to a pH of about 3 to about 9, and in another embodiment from about 4 to about 8. Nonlimiting examples of the acid include hydrochloric acid, citric acid, aspartic acid, glutamic acid, carbonic acid, tatronic acid, malic acid, malonic acid, tartaric acid, adipic acid, phosphoric acid, phthalic acid, glycolic acid, lactic acid, succinic acid, acetic acid, sulfuric acid, boric acid, formic acid, and mixtures thereof. The acid is present in any amount that results in the desired pH. For example, about 0.5 wt. % to about 1.5 wt. % of 6N HCl can be included in the dispersion of the invention.

As previously described, the size of the liquid crystals that form in the dispersion of the invention is important, with liquid crystals that have small diameters (e.g., less than about 100 μm, in one embodiment less than about 20 μm) being highly desired. The size of the liquid crystals that form depends on the nature of the charge-charge interaction that occurs when the high charge density cationic polymer is introduced to the detersive surfactant. A rapid charge-charge interaction between the high charge density cationic polymer and detersive surfactant results in liquid crystals with undesirable, large diameters, while a slow charge-charge interaction results in liquid crystals with desirable, small diameters. One important process parameter the affects the rate of charge-charge interaction between the cationic polymer and detersive surfactant is the instantaneous weight ratio. The instantaneous weight ratio is the ratio of the weight of cationic polymer just before it is introduced to the detersive surfactant, to the total weight of the forming dispersion, at any given point in time during processing.

In some embodiments, the instantaneous weight ratio of cationic polymer to forming dispersion is about 1:10 to about 1:100, in one embodiment from about 1:12.5 to about 1:50, in another embodiment from about 1:15 to about 1:30. For example, at any point in time in a system that has an instantaneous weight ratio of about 1:20, means that about 1 wt. % of cationic polymer is to be added to about 20 wt. % of the forming dispersion. Before any cationic polymer has been added to the forming dispersion, the forming dispersion contains only surfactant. Thus, the instantaneous weight ratio simplifies to about 1 wt. % of the cationic polymer being added to about 20 wt. % of detersive surfactant. During the addition of additional cationic polymer to the forming dispersion, the instantaneous weight ratio is about 1 wt. % of cationic polymer being added to about 20 wt. % of the forming dispersion (e.g., detersive surfactant and cationic polymer that has already been added). Thus, adding the cationic polymer to the forming dispersion using an instantaneous weight ratio of about 1:10 will allow for faster processing times, but also faster charge-charge interaction. Adding the cationic polymer to the forming dispersion using an instantaneous weight ratio of about 1:100 will result in slower processing times, but also slower charge-charge interaction.

The energy of the forming dispersion during addition of the cationic polymer to the detersive surfactant also dictates the rate of charge-charge interaction that occurs. In some embodiments, the energy of the forming dispersion during addition of the cationic polymer to the detersive surfactant can be about 10 Joules per kilogram (J/kg) to about 10,000 J/kg, in one embodiment from about 100 J/kg to about 7,500 J/kg, in yet another embodiment from about 500 J/kg to about 5,000 J/kg. A high energy device can be used to slow the charge-charge interaction between the detersive surfactant and cationic polymer, and to break up any agglomerates that form. In one embodiment, the high energy device is selected from the group consisting of a high shear mixer, a static mixer, a prop mixer, an in-tank mixer, a rotor-stator mill, a rotor-stator power incorporation device, or a homogenizer. In some embodiments, the homogenizer is a Liquid Whistle SONOLATOR® from Sonic Corp of CT. The Liquid Whistle is a high pressure homogenizer that subjects fluids to high pressure, extreme acceleration and ultrasonic cavitation by forcing the material through an engineered orifice.

The dispersion of the invention can be stabilized using any suspending agent (i.e. stabilizer) known for use in hair care or personal care products, provided that the components are physically and chemically compatible with the essential components described herein, or do not otherwise unduly impair product stability, aesthetics or performance. Suspending agents useful herein include anionic polymers and nonionic polymers. Useful herein are vinyl polymers such as cross linked acrylic acid polymers with the CTFA name Carbomer.

Other optional suspending agents include crystalline suspending agents which can be categorized as acyl derivatives, long chain amine oxides, and mixtures thereof. These suspending agents are described in U.S. Pat. No. 4,741,855. In one embodiment these suspending agents include ethylene glycol esters of fatty acids having from about 16 to about 22 carbon atoms. In another embodiment the suspending agents are the ethylene glycol stearates, both mono and distearate, but particularly the distearate containing less than about 7% of the mono stearate.

Other suitable suspending agents include alkanol amides of fatty acids, in one embodiment having from about 16 to about 22 carbon atoms, in another embodiment having about 16 to 18 carbon atoms, in another embodiment including stearic monoethanolamide, stearic diethanolamide, stearic monoisopropanolamide and stearic monoethanolamide stearate.

Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents.

Other optional suspending agents include bacterial cellulose networks, as described in International Patent Publication No. 2009/101545, which is incorporated herein by reference.

The dispersion of the invention can further include a hydrotrope. The hydrotrope facilitates the solubilization of some surfactants into aqueous solutions. In some embodiments, the hydrotrope includes C₁₋₈alkyl carboxylates, C₁₋₈alkyl sulfates, C₁₋₈alkyl benzene sulfonates, halogen benzoates (e.g., chlorbenzoate), C₁₋₈alkyl naphthalene carboxylates, (e.g., hydroxyl naphthalene carboxylate), urea, ethoxylated sulfates, and mixtures thereof. The C₁₋₈alkyl benzene sulfonates can include C₁₋₈alkyl cumene sulfonates, C₁₋₈alkyl toluene sulfonates (e.g., para-toluene sulfonate), C₁-C₈alkyl xylene sulfonates, and mixtures thereof. For example, the hydrotrope can include, sodium xylene sulfonates, potassium xylene sulfonates, ammonium xylene sulfonates, calcium xylene sulfonates, sodium toluene sulfonates, potassium toluene sulfonates, sodium cumene sulfonates, ammonium cumene sulfonates, sodium alkyl naphthalene sulfonates, sodium butyl naphthalene sulfonates, and mixtures thereof. The hydrotrope can be present in any amount that is sufficient to help solubilize a surfactant. In some embodiments, the hydrotrope is present in an amount of about 0.5 wt. % to about 5.0 wt. %, in one embodiment from about 1.0 wt. % to about 3.0 wt. %, based on the total weight of the dispersion.

The dispersion of the invention can further include an electrolyte. The electrolyte can function to slow and weaken the charge-charge interaction between the detersive surfactant and cationic polymer. The electrolyte can be an inorganic salt or an organic salt. Generally, inorganic electrolytes are chosen over organic electrolytes for better weight efficiency and lower costs. Mixtures of inorganic and organic salts can be used. Typical levels of electrolyte in the compositions are less than about 10 wt. %, based on the total weight of the dispersion. In one embodiment, the levels of electrolytes in the dispersion is about 0.5 wt. % to about 5 wt. % by weight, based on the total weight of the dispersion, in yet another embodiment about 0.75 wt. % to about 2.5 wt. %, and in yet another embodiment from about 1 wt. % to about 2 wt. %, based on the total weight of the dispersion.

Nonlimiting examples of inorganic salts suitable for use in the dispersion of the invention include MgI₂, MgBr₂, MgCl₂, Mg(NO₃)₂, Mg₃(PO₄)₂, Mg₂P₂O₇, MgSO₄, magnesium silicate, NaI, NaBr, NaCl, NaF, Na₃(PO₄), NaSO₃, Na₂SO₄, Na₂SO₃, NaNO₃, NaIO₃, Na₃(PO₄), Na₄P₂O₇, sodium silicate, sodium metasilicate, sodium tetrachloroaluminate, sodium tripolyphosphate (STPP), Na₂Si₃O₇, sodium zirconate, CaF₂, CaCl₂, CaBr₂, CaI₂, CaSO₄, Ca(NO₃)₂, Ca, KI, KBr, KCl, KF, KNO₃, KIO₃, K₂SO₄, K₂SO₃, K₃(PO₄), K₄(P₂O₇), potassium pyrosulfate, potassium pyrosulfite, LiI, LiBr, LiCl, LiF, LiNO₃, AlF₃, AlCl₃, AlBr₃, AlI₃, Al₂(SO₄)₃, Al(PO₄), A(NO₃)₃, aluminum silicate; including hydrates of these salts and including combinations of these salts or salts with mixed cations e.g. potassium alum AlK(SO₄)₂ and salts with mixed anions, e.g. potassium tetrachloroaluminate and sodium tetrafluoroaluminate. Mixtures of above salts are also useful

Organic salts useful in this invention include, magnesium, sodium, lithium, potassium, zinc, and aluminum salts of the carboxylic acids including formate, acetate, proprionate, pelargonate, citrate, gluconate, lactate aromatic acids e.g., benzoates, phenolate and substituted benzoates or phenolates, such as phenolate, salicylate, polyaromatic acids terephthalates, and polyacids e.g. m oxylate, adipate, succinate, benzenedicarboxylate, benzenetricarboxylate. Other useful organic salts include carbonate and/or hydrogencarbonate (HCO₃⁻¹) when the pH is suitable, alkyl and aromatic sulfates and sulfonates e.g., sodium methyl sulfate, benzene sulfonates and derivatives such as xylene sulfonate, and amino acids when the pH is suitable. Electrolytes can comprise mixed salts of the above, salts neutralized with mixed cations such as potassium/sodium tartrate, partially neutralized salts such as sodium hydrogen tartrate or potassium hydrogen phthalate, and salts comprising one cation with mixed anions.

In another aspect, the invention relates to a method of making a personal care product. In this method, the dispersion of the invention is combined with a personal care composition that includes one or more of the following: conditioning agents, natural cationic deposition polymers, synthetic cationic deposition polymers, anti-dandruff agents, gel networks (e.g., fatty alcohol/surfactant networks), particles, suspending agents, paraffinic hydrocarbons, propellants, viscosity modifiers, dyes, non-volatile solvents, water soluble diluents, water insoluble diluents, pearlescent aids, foam boosters, additional surfactants or nonionic cosurfactants, pediculocides, pH adjusting agents, perfumes, preservatives, chelants, proteins, skin active agents, sunscreens, UV absorbers, vitamins, and mixtures thereof, to form a personal care product.

The dispersion of the invention can be added to the personal care composition at a concentration that will result in about 0.025 wt. % to about 5 wt. %, in one embodiment from about 0.1 wt. % to about 3 wt. %, in another embodiment from about 0.2 wt. % to about 1 wt. % of the cationic polymer, based on the weight of the personal care product. For example, about 2 wt. % to about 15 wt. %, in one embodiment from about 5 wt. % to about 10 wt %, of the dispersion of the invention can be added to a personal care composition to form a personal care product, based on the total weight of the personal care product.

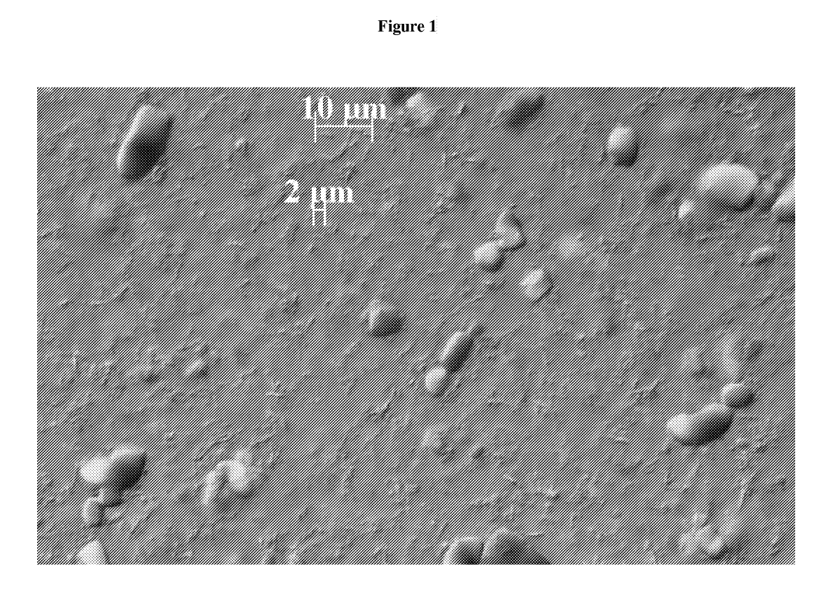

The dispersion of the invention can be added to the personal care composition by any means or method typically used to make personal care products. FIG. 1 shows an image of a personal care product, taken using Nomarski differential interference contrast (DIC) microscopy (Axioskop 2 plus scope, NeoFLUAR 40× objective lens, Axiocam HRC camera, Axiovision 3.1 photo software, all Zeiss brand), to which the dispersion of the invention has been incorporated. Samples were prepared by adding eight to ten microliters of the personal care product to a slide, which was mounted beneath a cover slip. The larger particles in the image (e.g., about 2 μm to about 10 μm in diameter) are liquid crystals of the cationic polymer, diallyldimethyl ammonium chloride (DADMAC, Polyquaternium-6) and a surfactant. The smaller, feather-like particles (i.e., about 2 μm in length) are ethylene glycol distearate (EGDS) crystals.

### 1. Conditioning Agents

a. Oily Conditioning Agent

In some embodiments, the dispersion of the invention is combined with a personal care composition that includes one or more oily conditioning agents to form a personal care product. Oily conditioning agents include materials which are used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. The oily conditioning agents useful in the compositions of the present invention typically comprise a water-insoluble, water-dispersible, non-volatile, liquid that forms emulsified, liquid particles. Suitable oily conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein.

One or more oily conditioning agents are typically present at a concentration of about 0.01 wt. % to about 10 wt. %, in one embodiment from about 0.1 wt. % to about 8 wt. %, in another embodiment from about 0.2 wt. % to about 4 wt. %, based on the weight of the personal care composition.

b. Silicone Conditioning Agent

In one embodiment the oily conditioning agents are a water-insoluble silicone conditioning agent. The silicone conditioning agent may comprise volatile silicone, non-volatile silicone, or combinations thereof. In one embodiment the conditioning agents are non-volatile silicone. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the personal care compositions p have a viscosity, as measured at 25° C. of from about 20 to about 2,000,000 centistokes (“csk”), in one embodiment from about 1,000 to about 1,800,000 csk, in yet another embodiment from about 5,000 to about 1,500,000 csk, in even yet another embodiment from about 10,000 to about 1,000,000 csk.

In an opaque composition embodiment of the present invention, the personal care composition comprises a non-volatile silicone oil having a particle size as measured in the personal care composition of about 1 μm to about 50 μm. In an embodiment of the present invention for small particle application to the hair, the personal care composition comprises a non-volatile silicone oil having a particle size as measured in the personal care composition from about 100 nm to about 1 μm. A substantially clear composition embodiment of the present invention comprises a non-volatile silicone oil having a particle size as measured in the personal care composition of less than about 100 nm.

Non-volatile silicone oils suitable for use in the personal care compositions can be selected from organo-modified silicones and fluoro-modified silicones. In one embodiment of the present invention, the non-volatile silicone oil is an organo-modified silicone which comprises an organo group selected from the group consisting of alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups. In one embodiment of the present invention, the non-volatile silicone oil is dimethicone.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

Silicone fluids suitable for use in the personal care compositions are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984).

c. Organic Conditioning Oils

The oily conditioning agent of the personal care compositions can further include at least one organic conditioning oil, either alone or in combination with other conditioning agents, such as the silicones described above.

d. Hydrocarbon Oils

Suitable organic conditioning oils for use as conditioning agents in the personal care compositions include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils may be from about C₁₂ to about C₁₉. Branched chain hydrocarbon oils, including hydrocarbon polymers, typically will contain more than 19 carbon atoms.

Specific nonlimiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include 2,2,4,4,6,6,8,8-dimethyl-10-methylundec ane and 2,2,4,4,6,6-dimethyl-8-methylnonane, available from Permethyl Corporation. In one embodiment the hydrocarbon polymer is polybutene, such as the copolymer of isobutylene and butene, which is commercially available as L-14 polybutene from Amoco Chemical Corporation.

e. Polyolefins

Organic conditioning oils for use in the personal care compositions can also include liquid polyolefins, liquid poly-α-olefins, hydrogenated liquid poly-α-olefins. Polyolefins for use herein are prepared by polymerization of C₄ to about C₁₄ olefenic monomers, and in another embodiment from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents.

f. Fatty Esters

Other suitable organic conditioning oils for use as the conditioning agent in the personal care compositions include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

Examples of fatty esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate. Other fatty esters suitable for use in the compositions of the present invention are those known as polyhydric alcohol esters. Such polyhydric alcohol esters include alkylene glycol esters.

Still other fatty esters suitable for use in the personal care compositions are glycerides, including, but not limited to, mono-, di-, and tri-glycerides. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

g. Fluorinated Conditioning Compounds

Fluorinated compounds suitable for delivering conditioning to hair or skin as organic conditioning oils include perfluoropolyethers, perfluorinated olefins, fluorine based specialty polymers that may be in a fluid or elastomer form similar to the silicone fluids previously described, and perfluorinated dimethicones. Specific non-limiting examples of suitable fluorinated compounds include the Fomblin product line from Ausimont which includes HC/04, HC/25, HC01, HC/02, HC/03; Dioctyldodecyl Fluoroeptyl Citrate, commonly called Biosil Basics Fluoro Gerbet 3.5 supplied by Biosil Technologies; and Biosil Basics Fluorosil LF also supplied by Biosil Technologies.

h. Fatty Alcohols

Other suitable organic conditioning oils for use in the personal care compositions include, but are not limited to, fatty alcohols having at least about 10 carbon atoms, in one embodiment from about 10 to about 22 carbon atoms, in yet another embodiment from about 12 to about 16 carbon atoms. Also suitable for use in the personal care compositions of the present inventions are alkoxylated fatty alcohols which conform to the general formula:

CH₃(CH₂)ₙCH₂(OCH₂CH₂)ₚOH

wherein n is a positive integer having a value from about 8 to about 20, in one embodiment from about 10 to about 14, and p is a positive integer having a value from about 1 to about 30, in one embodiment from about 2 to about 23.

i. Alkyl Glucosides and Alkyl Glucoside Derivatives

Suitable organic conditioning oils for use in the personal care compositions include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

j. Quaternary Ammonium Compounds

Suitable quaternary ammonium compounds for use as conditioning agents in the personal care compositions include, but are not limited to, hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

Examples of other useful quaternary ammonium surfactants include, but are not limited to, Quaternium-33, Quaternium-43, isostearamidopropyl ethyldimonium ethosulfate, Quaternium-22 and Quaternium-26, or combinations thereof, as designated in the CTFA Dictionary.

Other hydrophilic quaternary ammonium compounds useful in a composition of the present invention include, but are not limited to, Quatemium-16, Quaternium-27, Quaternium-30, Quaternium-52, Quaternium-53, Quaternium-56, Quaternium-60, Quaternium-61, Quaternium-62, Quaternium-63, Quaternium-71, and combinations thereof.

k. Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof.

Glycerin may also be used as conditioning agent in the personal care compositions. In one embodiment of the present invention, glycerin may be present in a range of about 0.01 wt. % to about 10 wt. %, based on the total weight of the personal care product. In a further embodiment of the present invention, glycerin may be present in a range of about 0.1 wt. % to about 5 wt. %, based on the total weight of the personal care product. In yet a further embodiment of the present invention, glycerin may be present in a range of about 2 wt. % to about 4 wt. %, based on the total weight of the personal care product.

### 2. Additional Components

In some embodiments, the dispersion of the invention is combined with a personal care composition that includes one or more components known for use in hair care or personal care products, provided that the components are physically and chemically compatible with the essential components described herein, or do not otherwise unduly impair product stability, aesthetics or performance to form a personal care product. Individual concentrations of such additional components may range from about 0.001 wt. % to about 10 wt. %, based on the weight of the personal care product.

Non-limiting examples components that can be included in the personal care composition include: additional natural or synthetic cationic deposition polymers, anti-dandruff agents, gel networks (e.g., fatty alcohol/surfactant networks), particles, suspending agents, paraffinic hydrocarbons, propellants, viscosity modifiers, dyes, non-volatile solvents, water soluble diluents, water insoluble diluents, pearlescent aids, foam boosters, additional surfactants or nonionic cosurfactants, pediculocides, pH adjusting agents, perfumes, preservatives, chelants, proteins, skin active agents, sunscreens, UV absorbers, vitamins, and mixtures thereof.

a. Cellulose or Guar Cationic Deposition Polymers

The dispersion of the invention can be combined with personal care compositions that include cellulose or guar cationic deposition polymers to form a personal care product. Cellulose or glactomannan cationic deposition polymers are useful herein Generally, such cellulose or guar cationic deposition polymers may be present at a concentration from about 0.05 wt. % to about 5 wt. %, based on the total weight of the personal care product. Suitable cellulose or guar cationic deposition polymers have a molecular weight of greater than about 5,000. In one embodiment the cellulose or guar cationic deposition polymers have a molecular weight of greater than about 200,000. Additionally, such cellulose or guar deposition polymers have a charge density from about 0.15 meq/g to about 4.0 meq/g at the pH of intended use of the personal care product, which pH will generally range from about pH 3 to about pH 9, in one embodiment between about pH 4 and about pH 8. The pH of the compositions of the present invention are measured neat.

Suitable cellulose or guar cationic polymers include those which conform to the following formula:

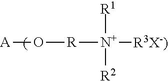

wherein A is an anhydroglucose residual group, such as a cellulose anhydroglucose residual; R is an alkylene oxyalkylene, polyoxyalkylene, or hydroxyalkylene group, or combination thereof, R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) in one embodiment being about 20 or less; and X is an anionic counterion. Non-limiting examples of such counterions include halides (e.g., chlorine, fluorine, bromine, iodine), sulfate and methylsulfate. The degree of cationic substitution in these polysaccharide polymers is typically from about 0.01 to about 1 cationic groups per anhydroglucose unit.

In one embodiment of the invention, the cellulose or guar cationic polymers are salts of hydroxyethyl cellulose reacted with trimethyl ammonium substituted epoxide, referred to in the industry (CTFA) as Polyquaternium 10 and available from Amerchol Corp. (Edison, N.J., USA).

Other suitable cationic deposition polymers include cationic guar gum derivatives, such as guar hydroxypropyltrimonium chloride, specific examples of which include the Jaguar series, in one embodiment Jaguar C-17®, commercially available from Rhone-Poulenc Incorporated, and in another embodiment including Jaguar C-500®, commercially available from Rhodia.

b. Synthetic Cationic Deposition Polymers

The dispersion of the invention can be combined with personal care compositions that include synthetic cationic deposition polymers to form a personal care product. Generally, such synthetic cationic deposition polymers may be present at a concentration from about 0.025 wt. % to about 5 wt. %, based on the total weight of the personal care product. Such synthetic cationic deposition polymers have a molecular weight from about 1,000 to about 5,000,000. Additionally, such synthetic cationic deposition polymers have a charge density from about 0.1 meq/g to about 5.0 meq/g.

Suitable synthetic cationic deposition polymers include those which are water-soluble or dispersible, cationic, non-crosslinked, conditioning copolymers comprising: (i) one or more cationic monomer units; and (ii) one or more nonionic monomer units or monomer units bearing a terminal negative charge; wherein said copolymer has a net positive charge, a cationic charge density of from about 0.5 meq/g to about 10 meg/g, and an average molecular weight from about 1,000 to about 5,000,000.

Non-limiting examples of suitable synthetic cationic deposition polymers are described in United States Patent Application Publication US 2003/0223951 A1 to Geary et al.

c. Anti-Dandruff Actives

The dispersion of the invention can be combined with personal care compositions that include an anti-dandruff agent to form a personal care product. Suitable, nonlimiting examples of anti-dandruff actives include: pyridinethione salts, zinc carbonate, azoles, such as ketoconazole, econazole, and elubiol, selenium sulfide, particulate sulfur, salicylic acid and mixtures thereof. A typical anti-dandruff particulate is a pyridinethione salt. Such anti-dandruff particulate should be physically and chemically compatible with the components of the dispersion, and should not otherwise unduly impair product stability, aesthetics or performance.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Azole anti-microbials include imidazoles such as climbazole and ketoconazole.

Selenium sulfide compounds are described, for example, in U.S. Pat. No. 2,694,668; U.S. Pat. No. 3,152,046; U.S. Pat. No. 4,089,945; and U.S. Pat. No. 4,885,107.

Sulfur may also be used as a particulate anti-microbial/anti-dandruff agent in the anti-microbial compositions of the present invention.

The present invention may further comprise one or more keratolytic agents such as Salicylic Acid.

Additional anti-microbial actives of the present invention may include extracts of melaleuca (tea tree) and charcoal.

When present in the composition, the anti-dandruff active is included in an amount of about 0.01 wt. % to about 5 wt. %, in one embodiment from about 0.1 wt. % to about 3 wt. %, and in another embodiment from about 0.3 wt. % to about 2 wt. %, based on the weight of the personal care product.

d. Particles

In some embodiments, the dispersion of the invention can be combined with a personal care composition that includes particles to form a personal care product. In one embodiment particles useful in the present invention are dispersed water-insoluble particles. Particles useful in the present invention can be inorganic, synthetic, or semi-synthetic. Compositions of the present invention typically incorporate no more than about 20 wt. %, in one embodiment no more than about 10 wt. % and in yet another embodiment no more than 2 wt. %, by weight of the personal care product, of particles. In an embodiment of the present invention, the particles have an average mean particle size of less than about 300 μm.

Non-limiting examples of inorganic particles include colloidal silicas, fumed silicas, precipitated silicas, silica gels, magnesium silicate, glass particles, talcs, micas, sericites, and various natural and synthetic clays including bentonites, hectorites, and montmorillonites.

Examples of synthetic particles include silicone resins, poly(meth)acrylates, polyethylene, polyester, polypropylene, polystyrene, polyurethane, polyamide (e.g., Nylon®), epoxy resins, urea resins, acrylic powders, and the like.

Non-limiting examples of hybrid particles include sericite & crosslinked polystyrene hybrid powder, and mica and silica hybrid powder.

e. Opacifying Agents

In some embodiments, the dispersion of the invention can be combined with a personal care composition that includes one or more opacifying agents. Opacifying agents are typically used in cleansing compositions to impart desired aesthetic benefits to the composition, such as color. One embodiment of the present invention includes opacifying agents at no more than about 20 wt. %, in another embodiment no more than about 10 wt. % and in yet another embodiment no more than 2 wt. %, based on the weight of the personal care product.

Suitable opacifying agents include, for example, fumed silica, polymethylmethacrylate, micronized Teflon®, boron nitride, barium sulfate, acrylate polymers, aluminum silicate, aluminum starch octenylsuccinate, calcium silicate, cellulose, chalk, corn starch, diatomaceous earth, Fuller's earth, glyceryl starch, hydrated silica, magnesium carbonate, magnesium hydroxide, magnesium oxide, magnesium trisilicate, maltodextrin, microcrystalline cellulose, rice starch, silica, titanium dioxide, zinc laurate, zinc myristate, zinc neodecanoate, zinc rosinate, zinc stearate, polyethylene, alumina, attapulgite, calcium carbonate, calcium silicate, dextran, nylon, silica silylate, silk powder, soy flour, tin oxide, titanium hydroxide, trimagnesium phosphate, walnut shell powder, or mixtures thereof. The above mentioned powders may be surface treated with lecithin, amino acids, mineral oil, silicone oil, or various other agents either alone or in combination, which coat the powder surface and render the particles hydrophobic in nature.

The opacifying agents may also comprise various organic and inorganic pigments. The organic pigments are generally various aromatic types including azo, indigoid, triphenylmethane, anthraquinone, and xanthine dyes. Inorganic pigments include iron oxides, ultramarine and chromium or chromium hydroxide colors, and mixtures thereof.

f. Suspending Agents

In some embodiments, the dispersions of the invention can be combined with a personal care composition that includes a suspending agent (i.e. stabilizer) at concentrations effective for suspending water-insoluble material in dispersed form in the compositions or for modifying the viscosity of the composition, to form a personal care product. Such concentrations generally range from about 0.1 wt. % to about 10 wt. %, in one embodiment from about 0.3 wt. % to about 5.0 wt. %, based on the total weight of the personal care product, of suspending agent.

Suspending agents useful herein include anionic polymers and nonionic polymers. Useful herein are vinyl polymers such as cross linked acrylic acid polymers with the CTFA name Carbomer.

Other optional suspending agents include crystalline suspending agents which can be categorized as acyl derivatives, long chain amine oxides, and mixtures thereof. These suspending agents are described in U.S. Pat. No. 4,741,855. These suspending agents include ethylene glycol esters of fatty acids having from about 16 to about 22 carbon atoms. In one embodiment the suspending agents are the ethylene glycol stearates, both mono and distearate, but particularly the distearate containing less than about 7% of the mono stearate.

Other suitable suspending agents include alkanol amides of fatty acids, in one embodiment having from about 16 to about 22 carbon atoms, in another embodiment from about 16 to 18 carbon atoms, examples of which include stearic monoethanolamide, stearic diethanolamide, stearic monoisopropanolamide and stearic monoethanolamide stearate.

Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents.

g. Paraffinic Hydrocarbons

In some embodiments, the dispersion of the invention can be combined with a personal care composition that includes one or more paraffinic hydrocarbons to form a personal care product. Paraffinic hydrocarbons suitable for use in compositions of the present invention include those materials which are known for use in hair care or other personal care compositions, such as those having a vapor pressure at 1 atm of equal to or greater than about 21° C. (about 70° F.). Non-limiting examples include pentane and isopentane.

h. Propellants

In some embodiments, the dispersion of the invention can be combined with a personal care composition that includes one or more propellants to form a personal care product. Propellants suitable for use in compositions of the present invention include those materials which are known for use in hair care or other personal care compositions, such as liquefied gas propellants and compressed gas propellants. Suitable propellants have a vapor pressure at 1 atm of less than about 21° C. (about 70° F.). Non-limiting examples of suitable propellants are alkanes, isoalkanes, haloalkanes, dimethyl ether, nitrogen, nitrous oxide, carbon dioxide, and mixtures thereof.

i. Other Optional Components

In some embodiments, the dispersion of the invention can be combined with a personal care composition that includes one or more fragrances to form a personal care product. The fragrances are used for aesthetic purposes and can be present in an amount of about 0.25 wt. % to about 2.5 wt. %, based on the total weight of the personal care product.

In some nonlimiting embodiments, the dispersion of the invention can be combined with a personal care composition that includes water-soluble and/or water-insoluble vitamins such as vitamins B1, B2, B6, B12, C, pantothenic acid, pantothenyl ethyl ether, panthenol, biotin and their derivatives, and vitamins A, D, E, and their derivatives, to form a personal care product. The dispersions of the present invention may also contain water-soluble and water-insoluble amino acids such as asparagine, alanine, indole, glutamic acid and their salts, and tyrosine, tryptamine, lysine, histadine and their salts.

In some embodiments the dispersion of the invention can be combined with a personal care composition that includes a mono- or divalent salt such as sodium chloride or other salts previously described herein to form a personal care product.

In some embodiments the dispersion of the invention can be combined with a personal care composition that includes chelating agents, e.g., EDTA, to form a personal care product. The chelating agent functions to potentiate preservatives and is present in an active amount of up to about 0.5 wt. %, based on the total weight of the personal care product.

In some embodiments the dispersion of the invention can be combined with a personal care composition that includes materials useful for hair loss prevention and hair growth stimulants or agents, to form a personal care product.

In some embodiments the dispersion of the invention can be combined with a personal care composition that includes one or more of viscosity modifiers, dyes, non-volatile solvents, water soluble diluents, water insoluble diluents, gel networks (e.g., fatty alcohol/surfactant networks), pearlescent aids, foam boosters, additional surfactants or nonionic cosurfactants, pediculocides, pH adjusting agents, preservatives, proteins, skin active agents, sunscreens, UV absorbers, or mixtures thereof, to form a personal care product.

In another aspect, the invention relates to a method of making a liquid cleansing product. In this method, the dispersion of the invention is combined with a liquid cleansing composition that includes one or more of the following: diamines, organic solvents, polycarboxylate polymers, magnesium ions, hydrotropes, polymer suds stabilizers, carboxylic acids, detersive enzymes, optical brighteners, dye transfer inhibition agents, suds suppressors, detersive soil release polymers, fabric care benefit agents, stabilizers, ancillary detersive surfactants, detersive builders, perfumes, coloring agents, enzymes, bleaches, mal-odor control agents, antimicrobials, anti-static agents, fabric softening agents, grease cleaning polymer, and mixtures thereof, as disclosed in International Patent Application Publication Number 2009/101545, to form a liquid cleansing product.

The liquid cleaning product can be any composition that has cleansing properties such as, for example, laundry detergents, dish detergents, shower gels, liquid hand cleansers, liquid dental compositions, facial cleansers, and fluids. The liquid cleansing product can also include fluids intended for impregnation into or on wiping articles (e.g., baby wipes).

The dispersion of the invention can be added to the liquid cleansing composition at a concentration that will result in about 0.025 wt. % to about 5 wt. %, in one embodiment from about 0.1 wt. % to about 3 wt. %, in another embodiment from about 0.2 wt. % to about 1 wt. % of the cationic polymer, based on the weight of the liquid cleansing product. For example, about 2 wt. % to about 15 wt. %, in one embodiment from about 5 wt. % to about 10 wt %, of the dispersion of the invention can be added to a liquid cleansing composition to form a liquid cleansing product, based on the total weight of the liquid cleansing product.

The dispersion of the invention can be added to the liquid cleansing composition by any means or method typically used to make liquid cleansing products.

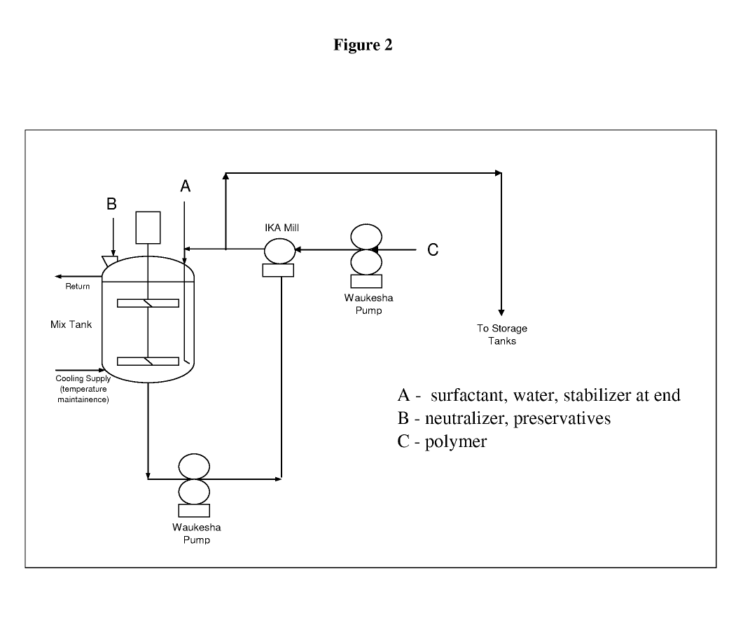

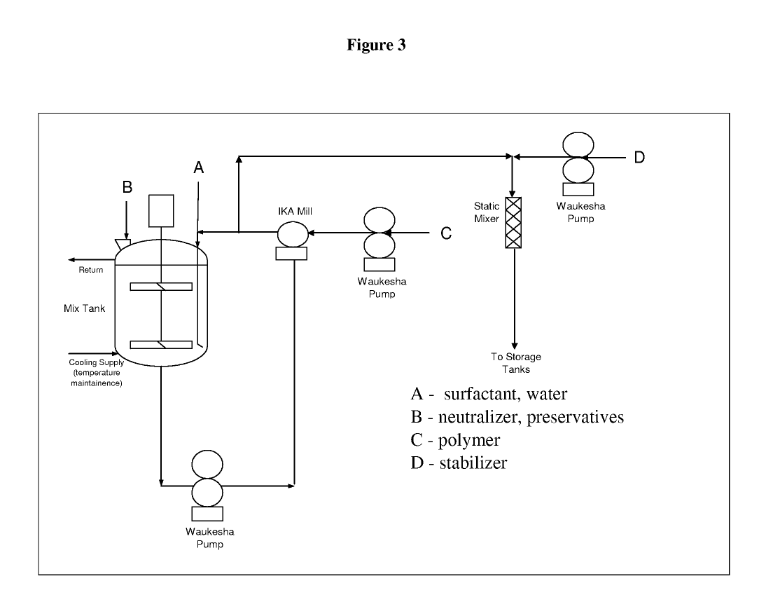

In one embodiment, the dispersions of the invention are produced using a high energy IKA mill, as depicted in FIG. 2. In this embodiment, detersive surfactant (e.g., SLS, SLE1S, SLE3S, or mixtures thereof) and water are introduced to the mix tank through dip tube A, and agitated at a rate sufficient to ensure good homogenization without causing aeration (e.g., 40 rpm to 50 rpm in a 130 L double impeller baffled tank). Preservatives (e.g. at least about 0.25 wt. % sodium benzoate, or about 5 ppm to about 15 ppm of methylchloroisothiazolinone (KATHON®), or mixtures thereof, based on the total weight of the composition) and acid (e.g., about 0.5 wt. % to about 1.5 wt. % of 6N HCl, based on the total weight of the composition) are added to the mix tank through addition port B and agitated (e.g., 40 rpm to 50 rpm in a 130 L double impeller baffled tank). The contents of the mix tank are pumped through a mill (e.g., an IKA mill) and re-enter the mix tank. This is referred to as a recirculating loop. The cationic polymer is added via addition port C and pumped through the mill, where it combines with the recirculating contents of the mix tank. The rate of addition is controlled to ensure the polymer stream to the recirculation stream is in an active weight ratio of no more than 1:15. Recirculation of the contents of the mix tank continues, with the cationic polymer added at the same ratio rate, until all of the cationic polymer has been incorporated. The number of times that recirculation occurs depends on the initial amount of cationic polymer that is added to the system. If about 3 wt. % of the cationic polymer is desired in the dispersion, about 2.5 to 3.0 cycles are required. After all of the cationic polymer has been incorporated, a suspending agent (e.g., about 6 wt. % EGDS crystallized out of a surfactant system) is added to the mix tank through dip tub A to stabilize the dispersion. The stabilized dispersion is then pumped out of the mix tank and to a storage tank. In some embodiments, the stabilizer is incorporated through a static mixer while being pumped to a storage tank (see FIG. 3).

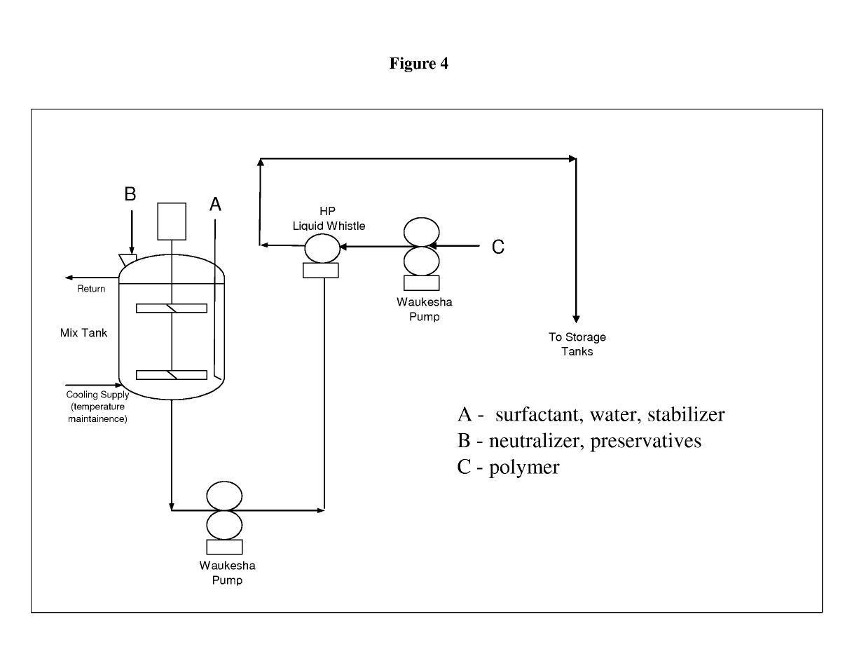

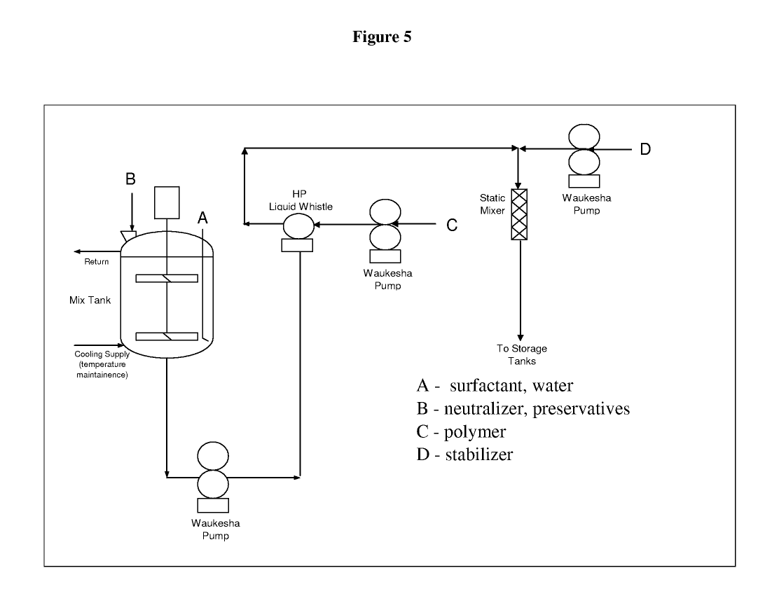

In another exemplary embodiment, the dispersions of the invention are produced using a Liquid Whistle SONOLATOR® from Sonic Corp of CT, as in FIG. 4. In this embodiment, detersive surfactant (e.g., SLS, SLE1S, SLE3S, or mixtures thereof) and water are introduced to the mix tank through dip tube A, and agitated at a rate sufficient to ensure good homogenization without causing aeration (e.g., 100 rpm in a 130 L double impeller baffled tank). Preservatives (e.g. at least about 0.25 wt. % sodium benzoate, or about 5 ppm to about 15 ppm of methylchloroisothiazolinone (KATHON®), or mixtures thereof, based on the total weight of the composition) and acid (e.g., about 0.5 wt. % to about 1.5 wt. % of 6N HCl, based on the total weight of the composition) are added to the mix tank through addition port B and agitated (e.g., 40 rpm to 50 rpm in a 130 L double impeller baffled tank). Following, a suspending agent (e.g., about 6 wt. % EGDS crystallized out of a surfactant system) is added to the mix tank through dip tub A and agitated. The contents of the mix tank are then pumped through the high pressure Liquid Whistle. Concurrently, the cationic polymer (e.g., DADMAC) is added via addition port C and pumped through the Liquid Whistle, where it combines with the contents of the mix tank. The resulting stabilized dispersion is then pumped to a storage tank. In some embodiments, the stabilizer is incorporated through a static mixer while being pumped to a storage tank (see FIG. 5).

### EXAMPLES

The dispersions described in the following Examples illustrate specific embodiments of the dispersions of the present invention, but are not intended to be limiting thereof. Other modifications can be undertaken by the skilled artisan without departing from the spirit and scope of this invention.

The dispersions described in the following Examples are prepared by conventional formulation and mixing methods, examples of which are described above All exemplified amounts are listed as weight percents and exclude minor materials such as diluents, preservatives, color solutions, imagery ingredients, botanicals, and so forth, unless otherwise specified.

The following examples are representative of suitable dispersion compositions of the invention.

| | | EXAMPLE | | | | | | | | | |
|---|---|---|---|---|---|---|---|---|---|---|---|
| Formula | | 1 | 2 | 3 | 4 | 5 | 6 | 7 | 8 | 9 | 10 |
| Sodium Lauryl Sulfate | A | 5.2 | 0.0 | 0.0 | 7.4 | 8.0 | 3.1 | 12.4 | 0.8 | 1.5 | 0.0 |
| Sodium Laureth (1) Sulfate | B | 10.5 | 0.0 | 0.0 | 3.3 | 0.0 | 4.2 | 0.0 | 17.3 | 4.5 | 21.0 |
| Sodium Laureth (3) Sulfate | C | 0.0 | 11.1 | 0.0 | 0.0 | 9.1 | 6.5 | 0.0 | 0.4 | 0.0 | 0.0 |
| Ammonium Cocoyl | D | 0.0 | 10.6 | 22.5 | 9.2 | 0.0 | 2.8 | 7.4 | 0.5 | 15.6 | 0.0 |
| Isethionate | | | | | | | | | | | |
| Diallyldimethyl ammonium | E | 4.0 | 1.8 | 0.0 | 2.7 | 1.6 | 2.6 | 2.1 | 0.0 | 0.0 | 3.0 |
| chloride (DADMAC, | | | | | | | | | | | |
| Polyquaternium-6) | | | | | | | | | | | |
| Polymethacrylamidopropyl | F | 0.0 | 1.8 | 2.6 | 0.0 | 2.1 | 0.2 | 1.2 | 2.6 | 4.0 | 0.0 |
| Trimonium Chloride | | | | | | | | | | | |
| Ethylene Glycol Distearate | G | 4.4 | 4.1 | 10.0 | 0.0 | 6.4 | 0.6 | 6.8 | 0.3 | 0.9 | 6.0 |
| Trihydroxystearin | H | 0.0 | 2.8 | 0.0 | 7.3 | 2.1 | 4.1 | 0.4 | 7.8 | 7.1 | 0.0 |
| Carbomer | I | 0.0 | 0.0 | 0.0 | 0.0 | 1.5 | 4.7 | 0.0 | 1.9 | 0.0 | 0.0 |
| Sodium Benzoate | J | 0.25 | 0.25 | 0.25 | 0.25 | 0.25 | 0.25 | 0.25 | 0.25 | 0.25 | 0.25 |
| Methylchloroisothiazolinone/ | K | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | L | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to |
| | | 100% | 100% | 100% | 100% | 100% | 100% | 100% | 100% | 100% | 100% |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Polyquaternium-6 available from Rhodia Inc. as Mirapol 100, 33% active
F Polymethacrylamidopropyl Trimonium Chloride available from Rhodia Inc. as Polycare 133, 33 wt. % active
G Ethylene Glycol Distearate available from Goldschmidt as Tegin BL315, 100 wt. % active
H Trihydroxystearin available from Elementis as Thixcin R, 100 wt. % active
I Carbomer available from Lubrizol Advanced Materials as Carbopol Ultrez 10, 100 wt. % active
J Sodium Benzoate available from DSM Special Products as Sodium Benzoate, 100 wt. % active
K Methylchloroisothiazolinone/Methylisothiazolinone available from Rohm & Haas as Kathon CG, 1.5 wt. % active (Listed as added wt. %, not as active wt. %)
L Hydrochloric Acid 6N available from Mallinckrodt Baker Inc. as Hydrochloric Acid 6N Solution (Listed as added wt. %, not as active wt. %)

| | | EXAMPLE | | | | | | | | | |
|---|---|---|---|---|---|---|---|---|---|---|---|
| Formula | | 11 | 12 | 13 | 14 | 15 | 16 | 17 | 18 | 19 | 20 |
| Sodium Lauryl Sulfate | A | 0.0 | 7.9 | 0.0 | 0.0 | 8.6 | 0.0 | 0.0 | 0.0 | 8.7 | 0.0 |
| Sodium Laureth (1) Sulfate | B | 17.3 | 0.0 | 8.4 | 0.0 | 0.0 | 12.0 | 4.7 | 0.0 | 11.3 | 0.0 |
| Sodium Laureth (3) Sulfate | C | 0.0 | 11.4 | 11.5 | 18.6 | 0.0 | 3.4 | 11.9 | 20.5 | 0.0 | 10.3 |
| Ammonium Cocoyl | D | 0.0 | 0.0 | 0.0 | 0.0 | 13.6 | 0.0 | 1.7 | 0.5 | 0.0 | 9.5 |
| Isethionate | | | | | | | | | | | |
| Diallyldimethyl ammonium | E | 2.0 | 3.0 | 0.0 | 0.0 | 2.2 | 2.6 | 1.6 | 3.2 | 2.6 | 0.0 |
| chloride (DADMAC, | | | | | | | | | | | |
| Polyquaternium-6) | | | | | | | | | | | |
| Polymethacrylamidopropyl | F | 0.7 | 0.3 | 3.6 | 2.7 | 1.6 | 0.0 | 2.1 | 0.0 | 0.0 | 3.3 |
| Trimonium Chloride | | | | | | | | | | | |
| Ethylene Glycol Distearate | G | 5.7 | 0.0 | 7.7 | 0.2 | 4.0 | 4.5 | 0.0 | 1.6 | 0.0 | 2.2 |
| Trihydroxystearin | H | 0.0 | 0.0 | 1.5 | 7.5 | 0.0 | 5.5 | 9.0 | 4.9 | 0.0 | 1.6 |
| Carbomer | I | 0.0 | 6.9 | 0.0 | 0.3 | 4.6 | 0.0 | 1.0 | 2.8 | 4.5 | 6.2 |
| Sodium Benzoate | J | 0.25 | 0.25 | 0.25 | 0.25 | 0.25 | 0.25 | 0.25 | 0.25 | 0.25 | 0.25 |
| Methylchloroisothiazolinone/ | K | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | L | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to |
| | | 100% | 100% | 100% | 100% | 100% | 100% | 100% | 100% | 100% | 100% |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Polyquaternium-6 available from Rhodia Inc. as Mirapol 100, 33% active
F Polymethacrylamidopropyl Trimonium Chloride available from Rhodia Inc. as Polycare 133, 33 wt. % active
G Ethylene Glycol Distearate available from Goldschmidt as Tegin BL315, 100 wt. % active
H Trihydroxystearin available from Elementis as Thixcin R, 100 wt. % active
I Carbomer available from Lubrizol Advanced Materials as Carbopol Ultrez 10, 100 wt. % active
J Sodium Benzoate available from DSM Special Products as Sodium Benzoate, 100 wt. % active
K Methylchloroisothiazolinone/Methylisothiazolinone available from Rohm & Haas as Kathon CG, 1.5 wt. % active (Listed as added wt. %, not as active wt. %)
L Hydrochloric Acid 6N available from Mallinckrodt Baker Inc. as Hydrochloric Acid 6N Solution (Listed as added wt. %, not as active wt. %)

| | | EXAMPLE | | | | | | | | | |
|---|---|---|---|---|---|---|---|---|---|---|---|
| Formula | | 21 | 22 | 23 | 24 | 25 | 26 | 27 | 28 | 29 | 30 |
| Sodium Lauryl Sulfate | A | 16.5 | 17.4 | 0.0 | 1.0 | 0.0 | 12.8 | 0.0 | 6.0 | 21.8 | 0.0 |
| Sodium Laureth (1) Sulfate | B | 0.7 | 0.0 | 8.6 | 0.0 | 0.0 | 0.0 | 0.0 | 0.0 | 0.0 | 0.0 |
| Sodium Laureth (3) Sulfate | C | 0.0 | 0.0 | 0.0 | 20.0 | 6.7 | 6.8 | 0.0 | 15.0 | 0.0 | 0.0 |
| Ammonium Cocoyl | D | 0.0 | 0.0 | 7.7 | 0.0 | 10.8 | 0.0 | 20.2 | 0.0 | 0.0 | 20.9 |
| Isethionate | | | | | | | | | | | |
| Diallyldimethyl ammonium | E | 1.7 | 0.9 | 0.9 | 0.0 | 3.4 | 2.7 | 2.8 | 3.0 | 2.2 | 4.0 |
| chloride (DADMAC, | | | | | | | | | | | |
| Polyquaternium-6) | | | | | | | | | | | |
| Polymethacrylamidopropyl | F | 1.0 | 3.0 | 1.7 | 4.0 | 0.0 | 0.0 | 0.0 | 0.0 | 0.7 | 0.0 |
| Trimonium Chloride | | | | | | | | | | | |
| Ethylene Glycol Distearate | G | 0.0 | 0.0 | 0.5 | 0.0 | 0.0 | 3.3 | 3.8 | 6.0 | 5.2 | 5.2 |
| Trihydroxystearin | H | 10.0 | 2.0 | 2.0 | 3.3 | 0.0 | 0.7 | 0.0 | 0.0 | 1.3 | 4.8 |
| Carbomer | I | 0.0 | 3.2 | 1.5 | 4.7 | 4.6 | 0.0 | 4.7 | 0.0 | 3.5 | 0.0 |
| Sodium Benzoate | J | 0.25 | 0.25 | 0.25 | 0.25 | 0.25 | 0.25 | 0.25 | 0.25 | 0.25 | 0.25 |
| Methylchloroisothiazolinone/ | K | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | L | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to |
| | | 100% | 100% | 100% | 100% | 100% | 100% | 100% | 100% | 100% | 100% |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Polyquaternium-6 available from Rhodia Inc. as Mirapol 100, 33% active
F Polymethacrylamidopropyl Trimonium Chloride available from Rhodia Inc. as Polycare 133, 33 wt. % active
G Ethylene Glycol Distearate available from Goldschmidt as Tegin BL315, 100 wt. % active
H Trihydroxystearin available from Elementis as Thixcin R, 100 wt. % active
I Carbomer available from Lubrizol Advanced Materials as Carbopol Ultrez 10, 100 wt. % active
J Sodium Benzoate available from DSM Special Products as Sodium Benzoate, 100 wt. % active
K Methylchloroisothiazolinone/Methylisothiazolinone available from Rohm & Haas as Kathon CG, 1.5 wt. % active (Listed as added wt. %, not as active wt. %)
L Hydrochloric Acid 6N available from Mallinckrodt Baker Inc. as Hydrochloric Acid 6N Solution (Listed as added wt. %, not as active wt. %)

| | | EXAMPLE | | | | | | | | | |
|---|---|---|---|---|---|---|---|---|---|---|---|
| Formula | | 31 | 32 | 33 | 34 | 35 | 36 | 37 | 38 | 39 | 40 |
| Sodium Lauryl Sulfate | A | 19.1 | 0.0 | 0.0 | 0.0 | 20.0 | 0.0 | 0.0 | 0.0 | 12.7 | 9.0 |
| Sodium Laureth (1) Sulfate | B | 0.0 | 8.0 | 8.7 | 15.0 | 0.0 | 8.2 | 0.0 | 5.0 | 0.0 | 7.2 |
| Sodium Laureth (3) Sulfate | C | 0.0 | 0.0 | 0.0 | 0.0 | 0.0 | 0.0 | 12.0 | 11.2 | 0.0 | 0.0 |
| Ammonium Cocoyl | D | 2.8 | 11.1 | 9.6 | 0.0 | 0.0 | 12.7 | 5.1 | 0.0 | 9.4 | 0.0 |
| Isethionate | | | | | | | | | | | |
| Diallyldimethyl ammonium | E | 3.9 | 0.3 | 1.7 | 0.0 | 0.0 | 3.1 | 2.6 | 0.9 | 0.0 | 0.0 |
| chloride (DADMAC, | | | | | | | | | | | |
| Polyquaternium-6) | | | | | | | | | | | |
| Polymethacrylamidopropyl | F | 0.1 | 3.0 | 2.2 | 4.0 | 4.0 | 0.0 | 0.0 | 3.1 | 2.6 | 2.8 |
| Trimonium Chloride | | | | | | | | | | | |
| Ethylene Glycol Distearate | G | 1.4 | 0.0 | 8.2 | 0.0 | 10.0 | 4.3 | 10.0 | 0.0 | 0.0 | 6.7 |
| Trihydroxystearin | H | 2.6 | 0.0 | 0.0 | 6.8 | 0.0 | 1.7 | 0.0 | 0.0 | 1.4 | 0.0 |
| Carbomer | I | 0.0 | 6.7 | 1.7 | 2.7 | 0.0 | 0.0 | 0.0 | 10.0 | 2.6 | 1.6 |
| Sodium Benzoate | J | 0.25 | 0.25 | 0.25 | 0.25 | 0.25 | 0.25 | 0.25 | 0.25 | 0.25 | 0.25 |
| Methylchloroisothiazolinone/ | K | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | L | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to |
| | | 100% | 100% | 100% | 100% | 100% | 100% | 100% | 100% | 100% | 100% |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Polyquaternium-6 available from Rhodia Inc. as Mirapol 100, 33% active
F Polymethacrylamidopropyl Trimonium Chloride available from Rhodia Inc. as Polycare 133, 33 wt. % active
G Ethylene Glycol Distearate available from Goldschmidt as Tegin BL315, 100 wt. % active
H Trihydroxystearin available from Elementis as Thixcin R, 100 wt. % active
I Carbomer available from Lubrizol Advanced Materials as Carbopol Ultrez 10, 100 wt. % active
J Sodium Benzoate available from DSM Special Products as Sodium Benzoate, 100 wt. % active
K Methylchloroisothiazolinone/Methylisothiazolinone available from Rohm & Haas as Kathon CG, 1.5 wt. % active (Listed as added wt. %, not as active wt. %)
L Hydrochloric Acid 6N available from Mallinckrodt Baker Inc. as Hydrochloric Acid 6N Solution (Listed as added wt. %, not as active wt. %)

| | | EXAMPLE | | | | | | | | | |
|---|---|---|---|---|---|---|---|---|---|---|---|
| Formula | | 41 | 42 | 43 | 44 | 45 | 46 | 47 | 48 | 49 | 50 |
| Sodium Lauryl Sulfate | A | 5.7 | 8.3 | 13.3 | 0.0 | 0.0 | 0.0 | 0.0 | 5.8 | 8.5 | 0.8 |
| Sodium Laureth (1) Sulfate | B | 0.0 | 9.6 | 0.0 | 0.0 | 5.9 | 5.7 | 7.1 | 9.4 | 9.5 | 0.7 |
| Sodium Laureth (3) Sulfate | C | 7.5 | 2.2 | 8.2 | 19.2 | 11.0 | 7.7 | 4.3 | 3.6 | 0.0 | 0.9 |
| Ammonium Cocoyl | D | 4.1 | 0.0 | 0.0 | 0.0 | 1.4 | 5.4 | 9.4 | 0.0 | 0.0 | 15.4 |
| Isethionate | | | | | | | | | | | |
| Diallyldimethyl ammonium | E | 4.0 | 0.8 | 1.1 | 4.0 | 1.2 | 2.7 | 3.3 | 0.1 | 2.8 | 2.6 |
| chloride (DADMAC, | | | | | | | | | | | |
| Polyquaternium-6) | | | | | | | | | | | |
| Polymethacrylamidopropyl | F | 0.0 | 3.1 | 2.4 | 0.0 | 1.7 | 1.2 | 0.0 | 3.3 | 1.1 | 1.4 |
| Trimonium Chloride | | | | | | | | | | | |
| Ethylene Glycol Distearate | G | 0.8 | 2.7 | 0.0 | 6.8 | 3.7 | 0.0 | 0.0 | 0.0 | 3.1 | 0.5 |
| Trihydroxystearin | H | 6.4 | 0.0 | 9.5 | 0.0 | 0.6 | 2.9 | 0.0 | 4.2 | 6.4 | 4.3 |
| Carbomer | I | 0.0 | 5.3 | 0.1 | 0.2 | 5.1 | 3.2 | 10.0 | 0.0 | 0.0 | 4.5 |
| Sodium Benzoate | J | 0.25 | 0.25 | 0.25 | 0.25 | 0.25 | 0.25 | 0.25 | 0.25 | 0.25 | 0.25 |
| Methylchloroisothiazolinone/ | K | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | L | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to |
| | | 100% | 100% | 100% | 100% | 100% | 100% | 100% | 100% | 100% | 100% |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Polyquaternium-6 available from Rhodia Inc. as Mirapol 100, 33% active
F Polymethacrylamidopropyl Trimonium Chloride available from Rhodia Inc. as Polycare 133, 33 wt. % active
G Ethylene Glycol Distearate available from Goldschmidt as Tegin BL315, 100 wt. % active
H Trihydroxystearin available from Elementis as Thixcin R, 100 wt. % active
I Carbomer available from Lubrizol Advanced Materials as Carbopol Ultrez 10, 100 wt. % active
J Sodium Benzoate available from DSM Special Products as Sodium Benzoate, 100 wt. % active
K Methylchloroisothiazolinone/Methylisothiazolinone available from Rohm & Haas as Kathon CG, 1.5 wt. % active (Listed as added wt. %, not as active wt. %)
L Hydrochloric Acid 6N available from Mallinckrodt Baker Inc. as Hydrochloric Acid 6N Solution (Listed as added wt. %, not as active wt. %)

| | | EXAMPLE | | | | | | | | | |
|---|---|---|---|---|---|---|---|---|---|---|---|
| Formula | | 51 | 52 | 53 | 54 | 55 | 56 | 57 | 58 | 59 | 60 |
| Sodium Lauryl Sulfate | A | 0.0 | 0.0 | 9.8 | 3.3 | 8.9 | 1.0 | 3.0 | 0.0 | 7.7 | 0.0 |
| Sodium Laureth (1) Sulfate | B | 17.2 | 0.0 | 0.0 | 8.1 | 0.0 | 15.1 | 2.5 | 0.0 | 0.0 | 0.0 |
| Sodium Laureth (3) Sulfate | C | 1.7 | 16.8 | 0.0 | 6.6 | 1.4 | 0.0 | 6.5 | 0.0 | 13.6 | 4.2 |
| Ammonium Cocoyl | D | 0.0 | 0.0 | 7.9 | 0.0 | 9.5 | 0.6 | 6.7 | 20.7 | 0.0 | 18.0 |
| Isethionate | | | | | | | | | | | |
| Diallyldimethyl ammonium | E | 4.0 | 2.2 | 0.1 | 2.8 | 0.0 | 2.1 | 0.0 | 2.7 | 0.0 | 1.6 |
| chloride (DADMAC, | | | | | | | | | | | |
| Polyquaternium-6) | | | | | | | | | | | |
| Polymethacrylamidopropyl | F | 0.0 | 0.8 | 2.5 | 0.0 | 2.7 | 1.0 | 4.0 | 1.2 | 2.6 | 1.0 |
| Trimonium Chloride | | | | | | | | | | | |
| Ethylene Glycol Distearate | G | 0.0 | 1.4 | 0.0 | 0.4 | 5.3 | 2.9 | 2.6 | 0.0 | 5.8 | 0.0 |
| Trihydroxystearin | H | 5.4 | 2.2 | 0.0 | 4.8 | 4.7 | 0.0 | 0.0 | 4.0 | 0.0 | 5.4 |
| Carbomer | I | 2.6 | 0.4 | 10.0 | 1.7 | 0.0 | 7.1 | 1.4 | 0.0 | 1.8 | 4.6 |
| Sodium Benzoate | J | 0.25 | 0.25 | 0.25 | 0.25 | 0.25 | 0.25 | 0.25 | 0.25 | 0.25 | 0.25 |
| Methylchloroisothiazolinone/ | K | 0.033 | 0.033 | 0.033 | 0.033 | 0.033 | 0.033 | 0.033 | 0.033 | 0.033 | 0.033 |
| Methylisothiazolinone | | | | | | | | | | | |
| Hydrochloric Acid | L | as | as | as | as | as | as | as | as | as | as |
| | | needed | needed | needed | needed | needed | needed | needed | needed | needed | needed |
| Water | | QS to | QS to | QS to | QS to | QS to | QS to | QS to | QS to | QS to | QS to |
| | | 100% | 100% | 100% | 100% | 100% | 100% | 100% | 100% | 100% | 100% |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Polyquaternium-6 available from Rhodia Inc. as Mirapol 100, 33% active
F Polymethacrylamidopropyl Trimonium Chloride available from Rhodia Inc. as Polycare 133, 33 wt. % active
G Ethylene Glycol Distearate available from Goldschmidt as Tegin BL315, 100 wt. % active
H Trihydroxystearin available from Elementis as Thixcin R, 100 wt. % active
I Carbomer available from Lubrizol Advanced Materials as Carbopol Ultrez 10, 100 wt. % active
J Sodium Benzoate available from DSM Special Products as Sodium Benzoate, 100 wt. % active
K Methylchloroisothiazolinone/Methylisothiazolinone available from Rohm & Haas as Kathon CG, 1.5 wt. % active (Listed as added wt. %, not as active wt. %)
L Hydrochloric Acid 6N available from Mallinckrodt Baker Inc. as Hydrochloric Acid 6N Solution (Listed as added wt. %, not as active wt. %)

| | | EXAMPLE | | | | | |
|---|---|---|---|---|---|---|---|
| Formula | | 61 | 62 | 63 | 64 | 65 | 66 |
| Sodium Lauryl Sulfate | A | 0.0 | 18.9 | 0.0 | 0.0 | 8.7 | 12.1 |
| Sodium Laureth (1) Sulfate | B | 6.6 | 0.0 | 3.5 | 0.0 | 3.9 | 4.8 |
| Sodium Laureth (3) Sulfate | C | 13.6 | 0.0 | 0.0 | 1.3 | 5.3 | 0.0 |
| Ammonium Cocoyl | D | 0.0 | 0.2 | 13.8 | 16.6 | 3.0 | 2.3 |
| Isethionate | | | | | | | |
| Diallyldimethyl ammonium | E | 1.6 | 4.0 | 3.3 | 0.0 | 3.4 | 1.2 |
| chloride (DADMAC, | | | | | | | |
| Polyquaternium-6) | | | | | | | |
| Polymethacrylamidopropyl | F | 1.1 | 0.0 | 0.0 | 3.2 | 0.0 | 2.3 |
| Trimonium Chloride | | | | | | | |
| Ethylene Glycol Distearate | G | 0.0 | 1.8 | 0.0 | 3.6 | 8.7 | 0.0 |
| Trihydroxystearin | H | 1.0 | 0.7 | 10.0 | 3.1 | 0.0 | 4.0 |
| Carbomer | I | 3.0 | 7.5 | 0.0 | 0.0 | 1.3 | 5.7 |
| Sodium Benzoate | J | 0.25 | 0.25 | 0.25 | 0.25 | 0.25 | 0.25 |
| Methylchloroisothiazolinone/ | K | 0.033 | 0.033 | 0.033 | 0.033 | 0.033 | 0.033 |
| Methylisothiazolinone | | | | | | | |
| Hydrochloric Acid | L | as | as | as | as | as | as |
| | | needed | needed | needed | needed | needed | needed |
| Water | | QS to 100% | QS to 100% | QS to 100% | QS to 100% | QS to 100% | QS to 100% |

A Sodium Lauryl Sulfate available from Cognis Corp. as Standapol WAQ-LC, 29 wt. % active
B Sodium Laureth (1) Sulfate available from Cognis Corp. as Standapol ES-1, 25 wt. % active
C Sodium Laureth (3) Sulfate available from Cognis Corp. as Standapol ES-3, 28 wt. % active
D Ammonium Cocoyl Isethionate available from BASF Chemicals as Jordapon ACI 30G, 30 wt. % active
E Polyquaternium-6 available from Rhodia Inc. as Mirapol 100, 33% active
F Polymethacrylamidopropyl Trimonium Chloride available from Rhodia Inc. as Polycare 133, 33 wt. % active
G Ethylene Glycol Distearate available from Goldschmidt as Tegin BL315, 100 wt. % active
H Trihydroxystearin available from Elementis as Thixcin R, 100 wt. % active
I Carbomer available from Lubrizol Advanced Materials as Carbopol Ultrez 10, 100 wt. % active
J Sodium Benzoate available from DSM Special Products as Sodium Benzoate, 100 wt. % active
K Methylchloroisothiazolinone/Methylisothiazolinone available from Rohm & Haas as Kathon CG, 1.5 wt. % active (Listed as added wt. %, not as active wt. %)
L Hydrochloric Acid 6N available from Mallinckrodt Baker Inc. as Hydrochloric Acid 6N Solution (Listed as added wt. %, not as active wt. %)

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for forming a dispersion comprising:
(a) adding (i) a synthetic, cationic polymer having a cationic charge density of about 2 molar equivalents per gram (meq/g) to about 7 meq/g to (ii) a neutralized, preserved, detersive surfactant at a rate sufficient to result in an instantaneous weight ratio of cationic polymer to surfactant of a about 1:10 to about 1:100 to form (iii) a dispersion comprising liquid crystals having an average diameter of less than about 100 μm, the dispersion having an energy of about 10 Joules per kilogram (J/kg) to about 10,000 J/kg; and,
(b) stabilizing the dispersion from phase separation;
wherein the temperature during each step is about 20° C. to about 40° C.

2. The method of claim 1, wherein the instantaneous ratio is about 1:12.5 to about 1:50.

3. The method of claim 1, wherein the energy is generated from a high energy device selected from the group consisting of a high shear mixer, a static mixer, a prop mixer, an in-tank mixer, a rotor-stator mill, a rotor-stator power incorporation device, and a homogenizer.

4. The method of claim 1, wherein the synthetic cationic polymer has an average molecular weight of about 1,000 to about 5,000,000.

5. The method of claim 1, wherein the synthetic cationic polymer comprises monomers selected from the group consisting of dimethylaminoethyl(meth)acrylate, dimethylaminopropyl(meth)acrylate, ditertiobutylaminoethyl(meth)acrylate, dimethylaminomethyl(meth)acrylamide, dimethylaminopropyl(meth)acrylamide; ethylenimine, vinylamine, 2-vinylpyridine, 4-vinylpyridine, trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride, diallyldimethyl ammonium chloride, trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride, trimethyl ammonium propyl(meth)acrylamido chloride, and mixtures thereof.

6. The method of claim 5, wherein the synthetic cationic polymer is diallyldimethyl ammonium chloride (DADMAC).

7. The method of claim 1, wherein the detersive surfactant comprises at least one anionic surfactant, wherein the anionic surfactant has an ethoxylate level of about 0 to about 10 and an anion level of about 1 to about 6.

8. The method of claim 7, wherein the detersive surfactant comprises an anion selected from the group consisting of sulfates, sulfonates, sulfosuccinates, isethionates, carboxylates, phosphates, phosphonates, and mixtures thereof.

9. The method of claim 8, wherein the detersive surfactant comprises sodium lauryl sulfate having an active weight ratio to the cationic polymer 2.5:1.

10. The method of claim 8, wherein the detersive surfactant is selected from the group consisting of sodium laureth sulfate 1-ethoxylate, sodium laureth sulfate 3-ethoxylate, and mixtures thereof and has an active weight ratio to the cationic polymer of 3.5:1.

11. The method of claim 8, wherein the detersive surfactant further comprises a compound selected from the group consisting of amphoteric surfactants, zwitterionic surfactants, cationic surfactants, nonionic surfactants, and mixtures thereof.

12. The method of claim 1, wherein stabilizing comprises adding a suspending agent to the dispersion selected from the group consisting of ethylene glycol monostearate, ethylene glycol distearate, stearic monoethanolamide, stearic diethanolamide, stearic monoisopropanolamide stearic monoethanolamide stearate, and mixtures thereof.

13. The method of claim 12, wherein the suspending agent comprises an anionic surfactant.

14. The method of claim 1, wherein the stabilized dispersion comprises about 1.5 wt. % to about 9.0 wt. % of synthetic, cationic polymer and about 5 wt. % to about 30 wt. % of detersive surfactant, based on the total weight of the stabilized dispersion.

15. The method of claim 1, wherein the stabilized dispersion comprises an additive selected from the group consisting of conditioning agents, natural cationic deposition polymers, synthetic cationic deposition polymers, anti-dandruff agents, gel networks, particles, suspending agents, paraffinic hydrocarbons, propellants, viscosity modifiers, dyes, non-volatile solvents, water soluble diluents, water insoluble diluents, pearlescent aids, foam boosters, surfactants, pediculocides, pH adjusting agents, perfumes, preservatives, chelants, proteins, skin active agents, sunscreens, UV absorbers, vitamins, and mixtures thereof.

16. The method of claim 1 further comprising adding the stabilized dispersion to a personal care composition comprising a component selected from the group consisting of conditioning agents, natural cationic deposition polymers, synthetic cationic deposition polymers, anti-dandruff agents, gel networks, particles, suspending agents, paraffinic hydrocarbons, propellants, viscosity modifiers, dyes, non-volatile solvents, water soluble diluents, water insoluble diluents, pearlescent aids, foam boosters, surfactants, pediculocides, pH adjusting agents, perfumes, preservatives, chelants, proteins, skin active agents, sunscreens, UV absorbers, vitamins, and mixtures thereof to form a personal care product.

17. The method of claim 16, wherein the cationic polymer is present in the personal care product in an amount of about 0.025 wt. % to about 5 wt. %, based on the total weight of the personal care product.

18. A method of making a personal care product comprising: combining,
(a) a personal care composition comprising a component selected from the group consisting of conditioning agents, natural cationic deposition polymers, synthetic cationic deposition polymers, anti-dandruff agents, gel networks, particles, suspending agents, paraffinic hydrocarbons, propellants, viscosity modifiers, dyes, non-volatile solvents, water soluble diluents, water insoluble diluents, pearlescent aids, foam boosters, surfactants, pediculocides, pH adjusting agents, perfumes, preservatives, chelants, proteins, skin active agents, sunscreens, UV absorbers, vitamins, and mixtures thereof; with
(b) the stabilized dispersion of claim 1.

19. A method of making a liquid cleansing product comprising: combining,
(a) a liquid cleansing composition comprising a component selected from the group consisting of diamines, organic solvents, polycarboxylate polymers, magnesium ions, hydrotropes, polymer suds stabilizers, carboxylic acids, detersive enzymes, optical brighteners, dye transfer inhibition agents, suds suppressors, detersive soil release polymers, fabric care benefit agents, stabilizers, ancillary detersive surfactants, detersive builders, perfumes, coloring agents, enzymes, bleaches, mal-odor control agents, antimicrobials, anti-static agents, fabric softening agents, grease cleaning polymers, and mixtures thereof.
(b) the stabilized dispersion of claim 1.

20. The method of claim 19, wherein the liquid cleaning product is selected from the group consisting of laundry detergents, dish detergents, shower gels, liquid hand cleansers, liquid dental compositions, facial cleansers, and fluids.
